(19) United States
(12) Patent Application Publication
(10) Pub. No.: US 2022/0283170 A1
(43) Pub. Date: Sep. 8, 2022

(54) COMPOUND AND FLUORESCENTLY LABELED BIOLOGICAL SUBSTANCE USING THE SAME

(71) Applicant: FUJIFILM Corporation, Tokyo (JP)

(72) Inventors: Kousuke WATANABE, KANAGAWA (JP); Yoshinori KANAZAWA, KANAGAWA (JP); Ryo FUJIWARA, KANAGAWA (JP); Kenji SHIROKANE, KANAGAWA (JP); Hiroaki TANAKA, KANAGAWA (JP); Yuki ARAI, KANAGAWA (JP); Kazuoki KOMIYAMA, KANAGAWA (JP)

(73) Assignee: FUJIFILM Corporation, Tokyo (JP)

(21) Appl. No.: 17/742,378

(22) Filed: May 11, 2022

Related U.S. Application Data
(63) Continuation of application No. PCT/JP2020/047278, filed on Dec. 17, 2020.

(30) Foreign Application Priority Data
Dec. 19, 2019 (JP) 2019-229598

(51) Int. Cl. G01N 33/58; C09K 11/06; C09B 23/01; C09B 23/06; C09B 23/10

(52) U.S. Cl. CPC G01N 33/582; C09K 11/06; C09B 23/0091; C09B 23/06; C09B 23/107; C09K 2211/1018; G01N 2021/6439

(57) ABSTRACT

Provided are any one of the following compounds and a fluorescently labeled biological substance having this compound.

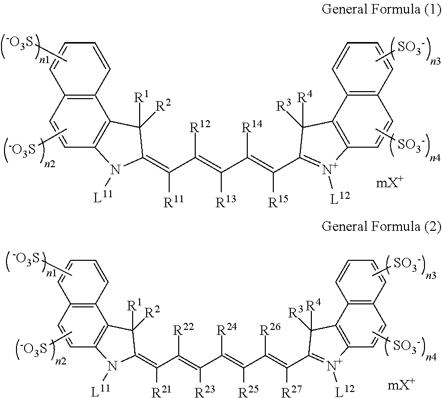

R¹ to R²⁷, L¹¹, L¹², and X⁺ represent a specific group, and at least one of R¹ to R⁴ represents a substituted alkyl group.

In a case where at least one of R¹ to R⁴ is an alkyl group having a carboxy group or a substituent capable of being bonded to a biological substance, at least one of R¹¹ to R²⁷ is an alkyl group or an aryl group, or at least one of R¹ to R⁴, which does not correspond to the alkyl group having a carboxy group or an alkyl group having a substituent capable of being bonded to a biological substance, is an alkyl group having a sulfoalkyl group through a single bond or a linking group.

The total of n1 to n4 is an integer of 3 or more, and m=n1+n2+n3+n4−1 is satisfied.

At least one of R¹ to R⁴, R¹³, L¹¹ or L¹² in Formula (1) and at least one of R¹ to R⁴, R²⁴, L¹¹, or L¹² in Formula (2) have a specific substituent.

### CROSS-REFERENCE TO RELATED APPLICATIONS

This application is a Continuation of PCT International Application No. PCT/JP2020/047278 filed on Dec. 17, 2020, which claims priority under 35 U.S.C. § 119 (a) to Japanese Patent Application No. 2019-229598 filed in Japan on Dec. 19, 2019. Each of the above applications is hereby expressly incorporated by reference, in its entirety, into the present application.

### BACKGROUND OF THE INVENTION

### 1. Field of the Invention

The present invention relates to a compound and a fluorescently labeled biological substance using the compound.

### 2. Description of the Related Art

In order to observe in vivo changes in response to various stimuli (diseases, environmental changes, and the like), fluorescently labeled biological substances obtained by labeling a biological substance (an antibody or the like) having affinity to a target substance to be detected, with a fluorescent compound (a dye), are often used.

For example, also in Western blotting (hereinafter, also abbreviated as WB) that detects a specific protein from a protein mixture, a fluorescence method in which the presence or absence or the abundance of the specific protein is detected using a fluorescently labeled antibody having affinity to this protein.

In addition, in bioimaging technology for analyzing the dynamics and functions of biological molecules, cells, tissues, and the like in a living body, in vivo fluorescence imaging by which a specific portion of a living body is capable of being visualized by fluorescence labelling is used as one of the technology for the living body observation.

A cyanine dye is known as a fluorescent dye that is used for fluorescence labelling. However, in a case where a cyanine dye is used for fluorescence labelling, interactions such as self-association between the dyes after labeling easily occur, and the fluorescence quantum yield tends to decrease.

As a technique for dealing with this problem, for example, WO2005/044923A discloses a cyanine dye having a sulfoalkyl group or a phosphate alkyl group as a substituent at the 3-position of the indolenine ring and further having a bonding group to a target material. Further, WO2002/026891A discloses a cyanine dye containing, as a substituent at the 3-position of the indolenine ring, a group that chemically reacts with a target material, such as a carboxyalkyl group or including a substance that has been conjugated.

According to WO2005/044923A and WO2002/026891A, the cyanine dyes described in the respective patent documents are said to exhibit high a fluorescence intensity as compared with the cyanine dyes in the related art by suppressing self-association between the dyes after labeling.

### SUMMARY OF THE INVENTION

In multicolor WB, a plurality of emission colors are detected in the range from the visible range to the near infrared range. As a result, it is necessary to select wavelengths so that the absorption and emission waveforms of a plurality of dyes have a suitable wavelength relationship so that crosstalk does not occur due to mutual interference in a case where the dyes are excited to emit light. Ideally, it should be adjusted so that only one dye emits light at one excitation light and the other dyes do not emit light. From this point of view, two kinds of excitation light sources having wavelengths separated to some extent, for example, around 700 nm and around 800 nm, are used for light emission in the near infrared range of the multicolor WB.

As compared with the detection by visible light excitation, the fluorescence detection by near-infrared light excitation can suppress the autofluorescence of the membrane, that is, the background fluorescence, and thus it is easy to increase the signal to noise ratio (the S/N ratio) and it is possible to detect a target protein with high sensitivity. As a result, in recent years, there has been an increasing need for fluorescence detection WB using light emission in the near infrared range in the analytical research on the trace amount of proteins.

However, in the near infrared range, the fluorescence quantum yield of the fluorescent dye is generally low, and thus it is difficult to obtain a high signal amount. In response to the request to observe or detect a protein with higher sensitivity, a sufficient fluorescence intensity cannot be obtained yet even with the fluorescence labelling using the cyanine dyes disclosed in WO2005/044923A and WO2002/026891A, and further improvement is still required.

An object of the present invention is to provide a compound that has a cyanine dye skeleton and exhibits an absorption wavelength peak suitable for color development in the near infrared range and an excellent fluorescence intensity. In addition, another object of the present invention is to provide a fluorescently labeled biological substance obtained by bonding the compound to a biological substance.

That is, the above objects of the present invention have been achieved by the following means.

[1] A compound represented by Formula (1) or Formula (2),

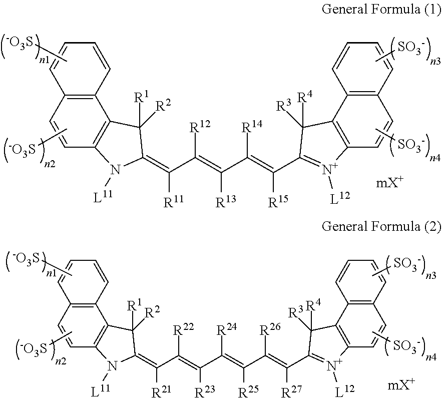

in the formulae, R¹ to R⁴ represent an alkyl group which may have a substituent,

provided that at least one of R¹ to R⁴ is an alkyl group having a substituent, R¹ and R² may be linked to each other to form a ring, and R³ and R⁴ may be linked to each other to form a ring,

R¹¹ to R¹⁵ and R²¹ to R²⁷ represent a hydrogen atom, an alkyl group, or an aryl group,

provided that in a case where at least one of R¹ to R⁴ is a carboxyalkyl group or an alkyl group having a substituent capable of being bonded to a biological substance, the following (I) or (II) is satisfied,

(I) in Formula (1), at least one of R¹¹ to R¹⁵ is an alkyl group or an aryl group and in Formula (2), at least one of R²¹ to R²⁷ is an alkyl group or an aryl group or

(II) at least one of R¹ to R⁴, which does not correspond to the carboxyalkyl group or the alkyl group having a substituent capable of being bonded to a biological substance, is an alkyl group having a sulfoalkyl group through a single bond or a linking group,

L¹¹ and L¹² represent an alkyl group which may have a substituent,

n1 to n4 are an integer of 0 to 2, where n1+n2≥1, n3+n4≥1, and n1+n2+n3+n4≥3 are satisfied,

m=n1+n2+n3+n4−1 is satisfied,

X⁺ represents a monovalent cation, and

at least one of R¹ to R⁴, R¹³, L¹¹, or L¹² in Formula (1) and at least one of R¹ to R⁴, R²⁴, L¹¹, or L¹² in Formula (2) have a carboxy group or a substituent capable of being bonded to a biological substance.

[2] The compound according to [1], in which the compound is represented by any one of Formulae (1-1) to (1-6) or Formulae (2-1) to (2-6),

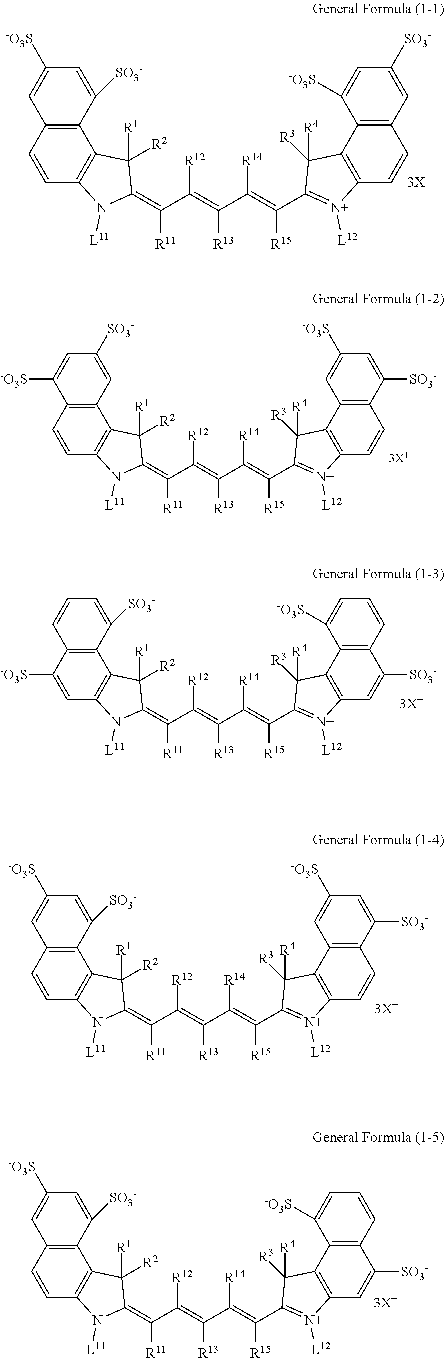

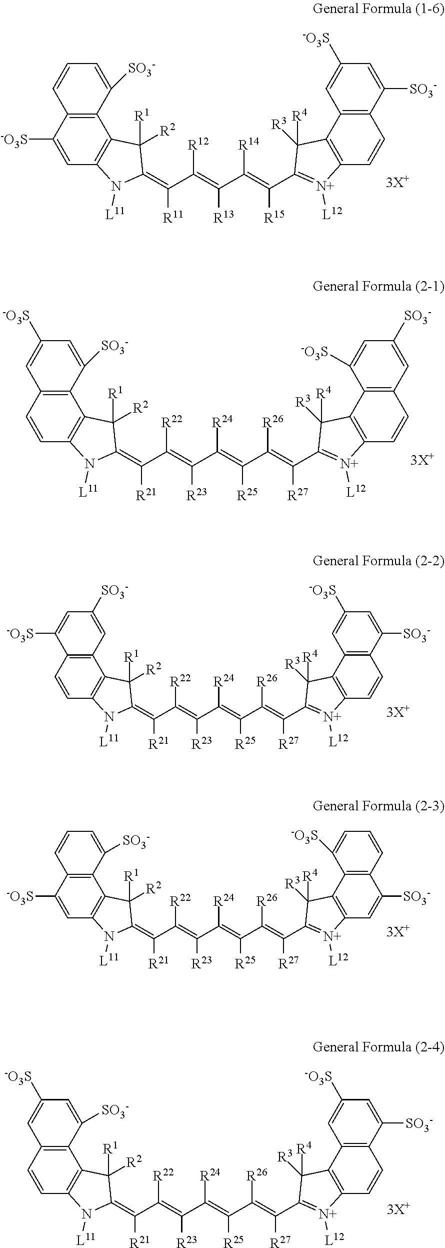

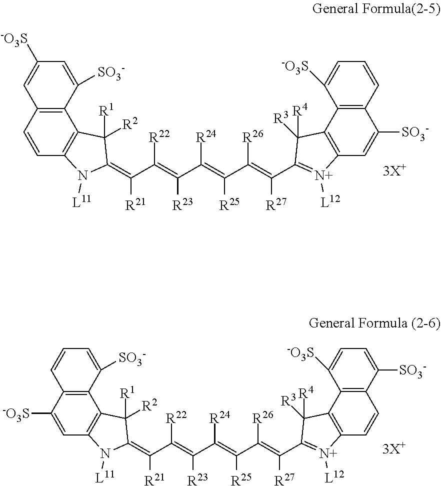

In the formula, R¹ to R⁴, R¹¹ to R¹⁵, R²¹ to R²⁷, L¹¹, L¹², and X⁺ are respectively synonymous with R¹ to R⁴, R¹¹ to R¹⁵, R²¹ to R²⁷, L¹¹, L¹², and X⁺ in Formula (1) and Formula (2).

[3] The compound according to [2], in which the compound is represented by any one of Formula (1-1), Formula (1-2), Formula (2-1), or Formula (2-2).

[4] The compound according to any one of [1] to [3], in which R¹ to R⁴ are an alkyl group which may have, as a substituent, a group selected from an alkoxy group, a carboxy group, an alkoxycarbonyl group, an acyloxy group, an aminocarbonyl group, an acylamino group, a sulfo group, and a phosphono group, and L¹¹ and L¹² are an alkyl group which may have, as a substituent, a group selected from an alkoxy group, a carboxy group, an alkoxycarbonyl group, an acyloxy group, an aminocarbonyl group, an acylamino group, a sulfo group, and a phosphono group.

[5] The invention according to any one of [1] to [4], in which R¹¹, R¹², R¹⁴, R¹⁵, R²¹ to R²³, and R²⁵ to R²⁷ are a hydrogen atom, and R¹³ and R²⁴ are a hydrogen atom or an alkyl group.

[6] The invention according to any one of [1] to [5], in which at least one of R¹ or R² is an alkyl group having a substituent, and at least one of R³ or R⁴ is an alkyl group having a substituent.

[7] The compound according to any one of [1] to [6], in which at least one of R¹ to R⁴ is a sulfoalkyl group,

provided that in a case where the sulfoalkyl group has no substituent other than the sulfo group, at least one of the alkyl groups having only this sulfo group is a branched sulfoalkyl group.

[8] The compound according to any one of [1] to [7], in which the at least one of R¹ to R⁴, R¹³, L¹¹, or L¹² in Formula (1) and the at least one of R¹ to R⁴, R²⁴, L¹¹ or L¹² in Formula (2) have a substituent capable of binding to an antibody.

[9] A fluorescently labeled biological substance that is obtained by bonding between the compound according to any one of [1] to [8] and a biological substance.

[10] The fluorescently labeled biological substance according to [9], in which the biological substance is any one of a protein, an amino acid, a nucleic acid, a sugar chain, a lipid, or a phospholipid.

The compound according to the aspect of the present invention has a cyanine dye skeleton and exhibits an absorption wavelength peak suitable for color development in the near infrared range and an excellent fluorescence intensity. Further, the fluorescently labeled biological substance according to the aspect of the present invention is labeled with the compound according to the aspect of the present invention and exhibits an absorption wavelength peak suitable for color development in the near infrared range and an excellent fluorescence intensity.

### DESCRIPTION OF THE PREFERRED EMBODIMENTS

In the present invention, in a case where there are a plurality of substituents or linking groups represented by a specific symbol or Formula (hereinafter, referred to as substituents or the like), or in a case where a plurality of substituents or the like are regulated at the same time, the substituents or the like may be the same or different from each other, unless otherwise specified. The same applies to the regulation of the number of substituents or the like. Further, in a case where a plurality of substituents or the like come close to each other (particularly in a case where they are adjacent to each other), they may be linked to each other to form a ring, unless otherwise specified. Further, unless otherwise specified, rings such as an alicyclic ring, an aromatic ring, and a heterocycle may be condensed to form a fused ring.

In the present specification, in a case where E type and Z type of the double bond are present in the molecule, any one of the E type or the Z type, or a mixture thereof may be used unless otherwise specified. In addition, in a case where a compound has a diastereomer and an enantiomer, any one of the diastereomer or the enantiomer may be used, or a mixture thereof may be used unless otherwise specified.

In the present invention, the representation of a compound or substituent is meant to include not only the compound itself but also a salt thereof, and an ion thereof. For example, the carboxy group, the sulfo group, and the phosphono group (—P(═O)(OH₂)) may have a salt structure by a hydrogen atom being dissociated therefrom. That is, in the present invention, the “carboxy group” is meant to include a salt of a carboxy group, the “sulfo group” is meant to include a salt of a sulfo group, and the “phosphono group” is meant to include a salt of a phosphono group. The monovalent cation in a case of constituting this salt structure has the same meaning as the description of X⁺ described later, which can be preferably applied.

In a case of a salt structure, the kind of the salt may be one kind, two or more kinds thereof may be mixed, a salt-type group and a group having a free acid structure may be mixed in a compound, and a compound having a salt structure and a compound having a free acid structure compound may be mixed. Further, in addition to the —SO₃⁻ group on the naphthalene ring in Formula (1) or (2), a dissociative substituent may be provided, and any dissociative substituent has a salt-type group.

In addition, it is meant to include those in which a part of the structure is changed within the scope that does not impair the effects of the present invention. Furthermore, it is meant that a compound, which is not specified to be substituted or unsubstituted, may have any substituent within the scope that does not impair the effects of the present invention. The same applies to a substituent (for example, a group represented by “alkyl group”, “methyl group”, “methyl”) and a linking group (for example, a group represented by “alkylene group”, “methylene group”, “methylene”). Among such substituents, a preferred substituent in the present invention is a substituent selected from a substituent group T described later.

In addition, in the present invention, the numerical range represented by using “to” means a range including the numerical values before and after “to” as the lower limit value and the upper limit value, respectively.

The compound according to an embodiment of the present invention is represented by Formula (1) or Formula (2). Although the details of the reason why the compound according to the embodiment of the present invention exhibits the excellent fluorescence intensity required for the multicolor WB are not clear, it is conceived as follows.

Each of the compounds according to the embodiment of the present invention has a cyanine skeleton in which nitrogen atoms in two benzoindolenine rings are linked by a polymethine chain, as represented by each formula. In addition, at least one of R¹ to R⁴, located at the 3-position in these two benzoindolenine rings, has an alkyl group having a substituent and at least one of R¹ to R⁴, R¹³, L¹¹, or L¹² in Formula (1) and at least one of R¹ to R⁴, R²⁴, L¹¹, or L¹² in Formula (2), located at the 3-position therein, have a substituent capable of being bonded to a biological substance. For this reason, it is conceived that in a substance obtained by bonding the compound according to the embodiment of the present invention to a biological substance, that is, in the fluorescently labeled biological substance according to the embodiment of the present invention, a substituent is present in a direction perpendicular to the plane of the cyanine dye skeleton, and thus the mutual interaction of the compounds is suppressed, whereby it is possible to suppress the decrease in fluorescence intensity due to the self-association of the compounds. Further, it is conceived that since the compound according to the embodiment of the present invention has one or more sulfo groups on each of the two benzoindolenine rings, three or more sulfo groups in total as the compound, sufficient hydrophilicity can be exhibited as well.

The compound according to the embodiment of the present invention, which is represented by Formula (1), has an excitation absorption wavelength in the vicinity of 685 nm, and the compound according to the embodiment of the present invention, which is represented by Formula (2), has an excitation absorption wavelength in the vicinity of 785 nm. As a result, the compounds respectively represented by Formulae (1) and (2) can be used as compounds that exhibit an excellent fluorescence intensity even in the multicolor WB having two kinds of light sources for excitation wavelengths, one in the vicinity of 700 nm and the other in the vicinity of 800 nm, respectively. At this point, the compound represented by Formula (1) or (2) is more convenient than the cyanine dye in the related art.

Hereinafter, the compound according to the embodiment of the present invention, which is represented by Formula (1) or Formula (2), will be described in detail.

<Compound Represented by Formula (1) or Formula (2)>

The compound according to the embodiment of the present invention, which is represented by Formula (1) or Formula (2), is as follows.

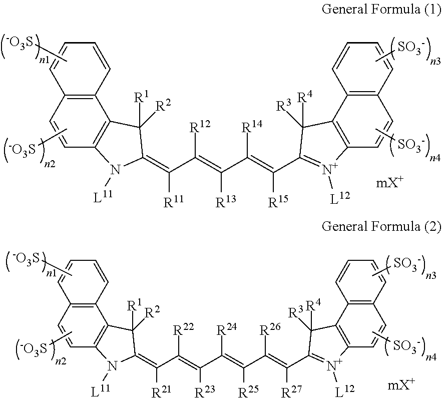

In the formulae, R¹ to R⁴ represent an alkyl group which may have a substituent. However, at least one of R¹ to R⁴ is an alkyl group having a substituent. R¹ and R² may be linked to each other to form a ring, and R³ and R⁴ may be linked to each other to form a ring.

R¹¹ to R¹⁵ and R²¹ to R²⁷ represent a hydrogen atom, an alkyl group, or an aryl group.

However, in a case where at least one of R¹ to R⁴ is a carboxyalkyl group or an alkyl group having a substituent capable of being bonded to a biological substance, the following (I) or (II) is satisfied.

(I) In Formula (1), at least one of R¹¹ to R¹⁵ is an alkyl group or an aryl group and in Formula (2), at least one of R²¹ to R²⁷ is an alkyl group or an aryl group.

(II) At least one of R¹ to R⁴, which does not correspond to the carboxyalkyl group or the alkyl group having a substituent capable of being bonded to a biological substance, is an alkyl group having a sulfoalkyl group through a single bond or a linking group.

L¹¹ and L¹² represent an alkyl group which may have a substituent.

n1 to n4 are an integer of 0 to 2, where n1+n2≥1, n3+n4≥1, and n1+n2+n3+n4≥3 are satisfied.

m=n1+n2+n3+n4−1 is satisfied.

X⁺ represents a monovalent cation.

A substituent may be provided on the naphthalene ring that can be substituted with a —SO₃⁻ group.

At least one of R¹ to R⁴, R¹³, L¹¹, or L¹² in Formula (1) and at least one of R¹ to R⁴, R²⁴, L¹¹, or L¹² in Formula (2) have a carboxy group or a substituent capable of being bonded to a biological substance.

It is conceived that in a case where R¹ to R⁴, R¹¹ to R¹⁵, and R²¹ to R²⁷ satisfy the regulation ((I) or (II)) including the above provisos, the compound represented by Formula (1) or (2) can achieve both the high fluorescence quantum yield obtained by the polycyclic fused-ring structure and the suppression of the intermolecular interaction that easily occurs due to the polycyclic fused-ring structure, and can exhibit an excellent fluorescence intensity.

Hereinafter, the substituent and the like in Formula (1) or Formula (2) will be described in detail.

(R¹ to R⁴)

R¹ to R⁴ each independently represent an alkyl group which may have a substituent. R¹ and R² may be linked to each other to form a ring, and R³ and R⁴ may be linked to each other to form a ring.

The alkyl group that can be adopted as R¹ to R⁴ is synonymous with an alkyl group in the substituent group T described later.

As long as at least one of R¹ to R⁴ is an alkyl group having a substituent, other substituents thereof may be each independently an unsubstituted alkyl group or may be an alkyl group having a substituent.

The unsubstituted alkyl group preferably has 1 to 6 carbon atoms, more preferably 1 to 4 carbon atoms, and still more preferably 1 or 2 carbon atoms.

The alkyl group having a substituent preferably has 1 to 10 carbon atoms, more preferably 1 to 8 carbon atoms, still more preferably 2 to 6 carbon atoms, and even still more preferably 3 to 5 carbon atoms. In addition, the number of atoms constituting the longest chain of the alkyl group having a substituent is preferably 3 to 12, more preferably 3 to 10, still more preferably 3 to 9, and particularly preferably 4 to 7.

It is conceived that in a case where the alkyl group having a substituent satisfies the above-described preferred number of carbon atoms or number of atoms, both excellent water solubility and suppression of intermolecular interaction can be achieved, and an excellent fluorescence intensity can be exhibited.

In the present invention, the “number of carbon atoms of an alkyl group having a substituent” means the number of carbon atoms including a substituent moiety. However, the number of carbon atoms in the substituent moiety capable of being bonded to a biological substance described later is not included.

In the present invention, “the number of atoms constituting the longest chain of an alkyl group having a substituent” means the number of atoms including a substituent moiety. It is noted that in a case where a substituent having a dissociative hydrogen atom such as a sulfo group or a carboxy group constitutes the longest chain, a group in which a hydrogen atom is dissociated does not include a dissociated hydrogen atom as an atom constituting the chain length, but a group in which a hydrogen atom is not dissociated includes a hydrogen atom as an atom constituting the chain length. In addition, the number of atoms in the substituent moiety capable of being bonded to a biological substance described later is not included.

Examples of the substituent which may be contained in the alkyl groups as R¹ to R⁴ include an alkoxy group, a carboxy group, an alkoxycarbonyl group, an acyloxy group, an aminocarbonyl group, an acylamino group, a sulfo group, and a phosphono group, as well as a group obtained by combining these substituents. In addition, examples thereof include a substituent capable of being bonded to a biological substance described later.

However, in a case where at least one of R¹ to R⁴ is a carboxyalkyl group or an alkyl group having a substituent capable of being bonded to a biological substance, the following (I) or (II) is satisfied. It is noted that both of the following (I) and (II) may be satisfied.

(I) In the compound represented by Formula (1), at least any one of R¹¹ to R¹⁵ is an alkyl group or an aryl group, and in the compound represented by Formula (2), at least any one of R²¹ to R²⁷ is an alkyl group or an aryl group.

(II) At least one of R¹ to R⁴, which does not correspond to the carboxyalkyl group or the alkyl group having a substituent capable of being bonded to a biological substance, is an alkyl group having a sulfoalkyl group through a single bond or a linking group.

The linking group in the above (II) is not particularly limited; however, preferred examples thereof include an ether bond, an ester bond, an amide bond.

In a case where at least any one of R¹ to R⁴ is a carboxyalkyl group or an alkyl group having a substituent capable of being bonded to a biological substance and does not satisfy at least any one of the above (I) or (II), the mutual association of dyes easily occurs, and thus an excellent fluorescence intensity cannot be obtained.

The alkyl group having a substituent, which is contained in at least one of R¹ to R⁴, is not particularly limited as long as it is an alkyl group having the above substituent; however, from the viewpoint of suppressing intermolecular interaction, it is preferably an alkyl group having, as a substituent, at least one of an alkoxy group, a carboxy group, a sulfo group, or a phosphono group, and more preferably an alkyl group having a sulfo group.

The alkyl group having the above substituent may have a substituent other than the alkoxy group, the carboxy group, the sulfo group, and the phosphono group. Further, it may be an alkyl group having an alkoxy group, a carboxy group, a sulfo group, or a phosphono group through a linking group (an ether bond, an ester bond, an amide bond, or the like) as an alkoxyalkyl group, a carboxyalkyl group, a sulfoalkyl group, or a phosphonoalkyl group, respectively.

In addition to the above, the above-described description of the number of carbon atoms of the alkyl group having a substituent and the number of atoms constituting the longest chain can be preferably applied to the number of carbon atoms and the number of atoms constituting the longest chain of the alkyl group having a substituent, where the alkyl group is contained in at least one of R¹ to R⁴. Since the substituents of R¹ to R⁴ protrude in a direction perpendicular to the benzoindolenine skeleton (plane), it is presumed that the larger the substituent, the more difficult the fused ring moiety undergo the π-π interaction (the association inhibitory effect becomes stronger), and thus the performance is improved.

In a case where at least one of R¹ to R⁴ is a sulfoalkyl group having no substituent other than the sulfo group, it is preferable that at least one of the alkyl groups having only this sulfo group is a branched sulfoalkyl group.

Here, the “branched sulfoalkyl group” means a form in which the molecular chain consisting of a carbon atom and a sulfur atom among the atoms constituting the sulfoalkyl group is not a linear chain but a branched chain.

Specifically, a from in which the molecular chain consisting of carbon atoms is a branched chain (however, a chain having a ring structure is included), or a form in which a sulfo group is attached to a carbon atom other than the terminal carbon atom in the molecular chain consisting of a carbon atom corresponds to the branched sulfoalkyl group. Further, a form in which R¹ and R² are linked to each other to form a ring and a form in which R³ and R⁴ are linked to each other to form a ring also correspond to the branched sulfoalkyl group.

In a case where at least one of R¹ to R⁴ is a sulfoalkyl group and this sulfoalkyl group is an alkyl group having only a sulfo group, it is conceived that in a case where the proviso that at least one of them is a branched sulfoalkyl group is satisfied, the intermolecular interaction of the compounds represented by Formula (1) or (2) can be further suppressed and an excellent fluorescence intensity can be exhibited.

In R¹ to R⁴, the number of alkyl groups having a substituent may be 1 or more, and it is preferably 1 to 3.

In a case where R¹ to R⁴ have neither a carboxy group nor a substituent capable of being bonded to a biological substance, it is preferable that at least one of R¹ or R² is an alkyl group having a substituent and that at least one of R³ or R⁴ is an alkyl group having a substituent, from the viewpoint of further improving the fluorescence intensity.

(R¹¹ to R¹⁵ and R²¹ to R²⁷)

R¹¹ to R¹⁵ and R²¹ to R²⁷ each independently represent a hydrogen atom, an alkyl group, or an aryl group.

The alkyl group and the aryl group, which can be adopted as R¹¹ to R¹⁵ and R²¹ to R²⁷, are synonymous with the alkyl group and the aryl group in the substituent group T described later, and the same applies to the preferred range thereof.

The alkyl group and the aryl group, which can be adopted as R¹¹ to R¹⁵ and R²¹ to R²⁷, may be each independently unsubstituted or have a substituent.

Examples of the substituent which may be contained in the alkyl group and the aryl group as R¹¹ to R¹⁵ and R²¹ to R²⁷ include a substituent in the substituent group T described later, and for example, an alkoxy group or a sulfo group is preferable. In addition, examples thereof include a substituent capable of being bonded to a biological substance described later.

Further, in a case where at least any one of R¹¹ to R¹⁵ or R²¹ to R²⁷ is an alkyl group having a substituent, the form of the above-described alkyl group having a substituent, which can be adopted by R¹ to R⁴, can be also preferably applied.

R¹¹, R¹², R¹⁴, R¹⁵, R²¹ to R²³, and R²⁵ to R²⁷ are preferably a hydrogen atom.

R¹³ and R²⁴ are preferably a hydrogen atom or an alkyl group.

(L¹¹ and L¹²)

L¹¹ and L¹² each independently represent an alkyl group which may have a substituent.

Examples of the substituent which may be contained in the alkyl groups as L¹¹ and L¹² include an alkoxy group, a carboxy group, an alkoxycarbonyl group, an acyloxy group, an aminocarbonyl group, an acylamino group, a sulfo group, and a phosphono group, as well as a group obtained by combining these substituents. In addition, examples thereof include a substituent capable of being bonded to a biological substance described later.

The alkyl group that can be adopted as L¹¹ and L¹² is synonymous with an alkyl group in the substituent group T described later.

The unsubstituted alkyl group preferably has 1 to 6 carbon atoms, more preferably 1 to 4 carbon atoms, and still more preferably 1 to 3 carbon atoms.

The number of carbon atoms contained in the alkyl group having a substituent is preferably 1 to 10, more preferably 1 to 8, still more preferably 1 to 7, even still more preferably 1 to 6, and even further still more preferably 1 to 5. In addition, the number of atoms constituting the longest chain of the alkyl group having a substituent is preferably 3 to 12, more preferably 3 to 10, and still more preferably 3 to 8.

The alkyl group having a substituent, as L¹¹ and L¹², is not particularly limited as long as it is an alkyl group having a substituent; however, from the viewpoint of further improving water solubility, it is preferably an alkyl group having, as a substituent, at least one of an alkoxy group, a carboxy group, a sulfo group, or a phosphono group, and more preferably an alkyl group having, as a substituent, at least one of a carboxy group or a sulfo group. Here, it may be an alkyl group having a substituent consisting of a combination of the above-described preferred substituents (the alkoxy group, the carboxy group, the sulfo group, and the phosphono group) and a group other than these substituents.

Further, in a case where at least any one of L¹¹ and L¹² is an alkyl group having a substituent, the form of the above-described alkyl group having a substituent, which can be adopted by R¹ to R⁴, can be also preferably applied.

In L¹¹ and L¹², the number of alkyl groups having a substituent is not particularly limited; however, it is preferably one or two, and more preferably two are more.

(X⁺)

X⁺ represents a monovalent cation.

The monovalent cation is not particularly limited, and examples thereof include alkali metal cations such as Na⁺, Li⁺, and K⁺, alkaline earth metal cations such as Mg²⁺, Ca²⁺, and Ba²⁺, and organic ammonium ions such as a trialkylammonium ion and a tetraalkylammonium ion.

(n1 to n4)

n1 to n4 are each independently an integer of 0 to 2, where n1+n2≥1, n3+n4≥1, and n1+n2+n3+n4≥3 are satisfied.

It is preferable that n1 and n3 are each independently 1 or 2.

It is preferable that n2 and n4 are each independently 0 or 1.

From the viewpoint of improving water solubility and suppressing association, n1+n2 and n3+n4 are each independently preferably an integer of 1 to 3, more preferably 1 or 2, and still more preferably 2.

n1+n2+n3+n4 is preferably an integer of 3 to 6, more preferably an integer of 3 to 5, still more preferably 3 or 4, and particularly preferably 4.

(m) m means the number of monovalent cations X⁺, by which the charge of the compound represented by Formula (1) or (2) is adjusted to become 0 as a whole.

That is, m satisfies m=n1+n2+n3+n4−1.

However, not all the cations contained in the compound represented by Formula (1) or (2) are denoted by mX⁺, and a cation which can be contained in a carboxy group, a sulfo group, or a phosphono group in the hydrophilic group described later is contained in the compound apart from this notation.

The naphthalene ring moiety (hereinafter, also simply referred to as a “naphthalene ring”) in the benzoindolenine ring that can be substituted with a —SO₃⁻ group may have a substituent other than the —SO₃⁻ group as long as the effects of the present invention are not impaired, and preferred examples thereof include a substituent in the substituent group T described later.

At least one of R₁ to R⁴, R¹³, L¹¹ or L¹² in Formula (1) and at least one of R¹ to R⁴, R²⁴, L¹¹, or L¹² in Formula (2) have a carboxy group or a substituent capable of being bonded to a biological substance described later.

The compound represented by Formula (1) or (2) can be bonded to a biological substance with the above-described carboxy group or a substituent capable of being bonded to a biological substance, whereby a targeted fluorescently labeled biological substance can be obtained. It is noted that as a substituent capable of being bonded to a biological substance, a carboxy group can be easily derived by a conventional method.

In the present invention, the “substituent capable of being bonded to a biological substance” includes a substituent capable of being bonded to a biological substance, derived from a carboxy group.

As described above, since the compound represented by Formula (1) or (2) is bonded to a biological substance with a substituent (specifically, R¹ to R⁴, R¹³, R²⁴, L¹¹, or L¹²) contained in a specific position in the cyanine skeleton structure, a fluorescently labeled biological substance to be obtained is conceived to exhibit an excellent fluorescence intensity, as described above.

It suffices that the number of groups having a carboxy group or a substituent capable of being bonded to a biological substance in R¹ to R⁴, R¹³, R²⁴, L¹¹, and L¹² is 1 or more in total, and it is preferably 1 to 3, more preferably 1 or 2, and still more preferably 1, from the viewpoint of the quantification of the substance to be detected.

From the viewpoint that sufficient hydrophilicity is imparted as a compound, the compound represented by Formula (1) or (2) preferably has 4 or more hydrophilic groups as the whole compound, more preferably has 4 to 8 hydrophilic groups as the whole compound, and still more preferably has 6 to 8 hydrophilic groups as the whole compound.

The hydrophilic group is not particularly limited, and examples thereof include an alkoxy group having a substituent, a carboxy group, a sulfo group, and a phosphono group.

That is, preferred examples of the form of the compound represented by Formula (1) or (2) also include those in which a hydrophilic group is contained in addition to the —SO₃⁻ group on the naphthalene ring. The position of the hydrophilic group other than the —SO₃⁻ group on the naphthalene ring is not particularly limited; however, it is preferable that, for example, at least any one of R¹ to R⁴, L¹¹, or L¹² is a substituent having a hydrophilic group.

<Compound Represented by Any of Formulae (1-1) to (1-6) or Formulae (2-1) to (2-6)>

The compound according to the embodiment of the present invention, which is represented by Formula (1), is preferably represented by any one of Formulae (1-1) to (1-6) and more preferably represented by any one of Formula (1-1) or Formula (1-2). Further, the compound according to the embodiment of the present invention, which is represented by Formula (2), is preferably represented by any one of Formulae (2-1) to (2-6) and more preferably represented by any one of Formula (2-1) or Formula (2-2).

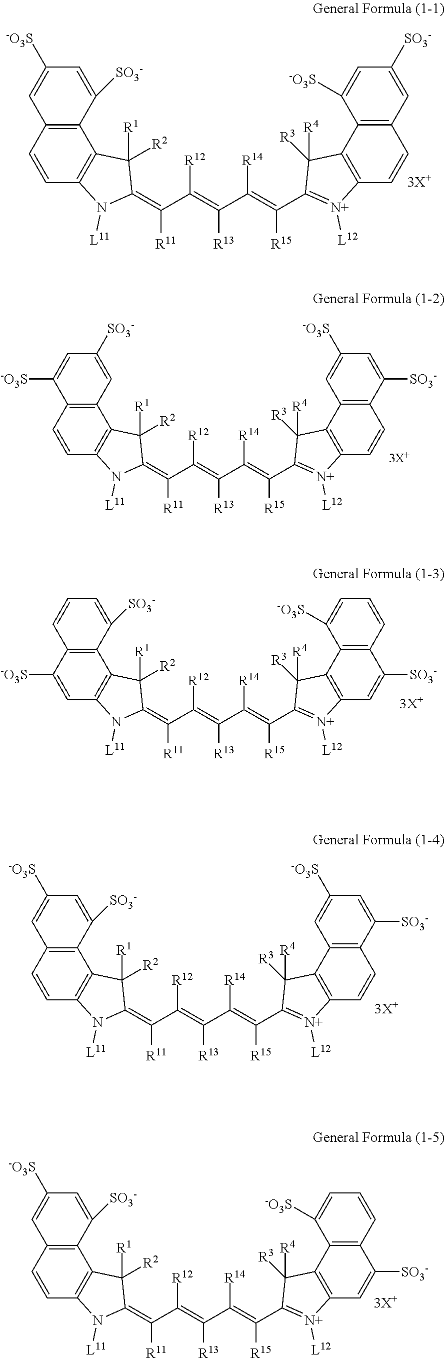

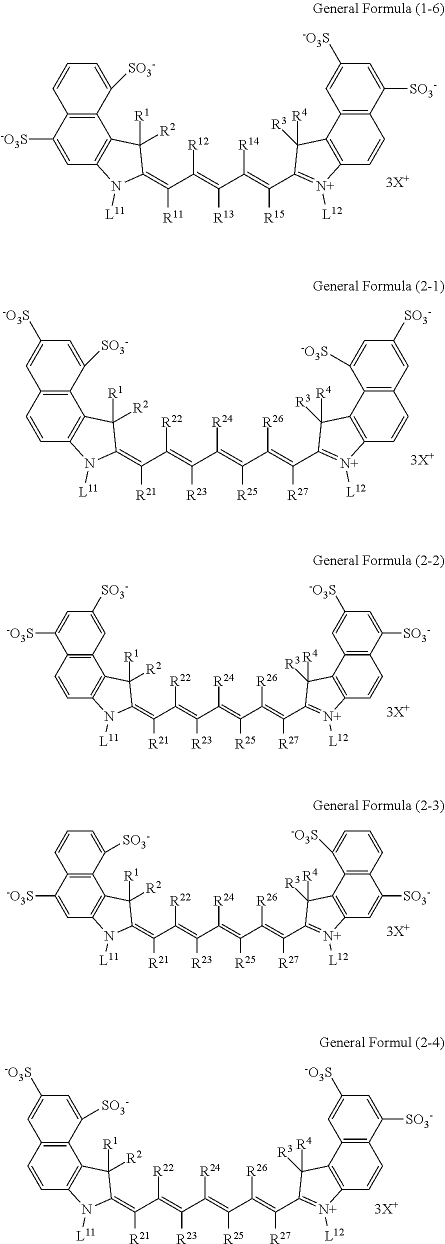

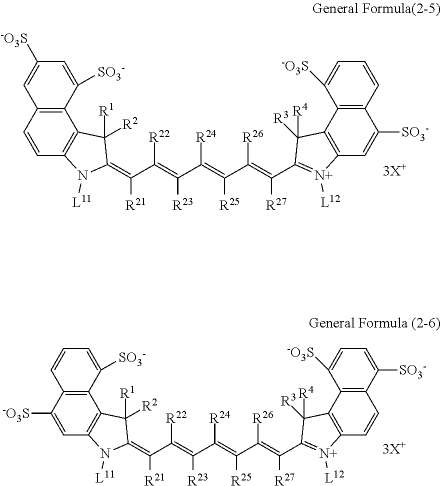

In the formula, R¹ to R⁴, R¹¹ to R¹⁵, R²¹ to R²⁷, L¹¹, L¹², and X⁺ are respectively synonymous with R¹ to R⁴, R¹¹ to R¹⁵, R²¹ to R²⁷, L¹¹, L¹², and X⁺ in Formula (1) and Formula (2), and the same applies to the preferred ones thereof.

Hereinafter, specific examples of the compound according to the embodiment of the present invention, which is represented by Formula (1) or Formula (2) and in which at least any one of R¹ to R⁴, R¹³, R²⁴, L¹¹, or L¹² has a carboxy group are shown below. However, the present invention is not limited to these compounds. In the following specific examples, a group having a dissociative hydrogen atom such as a carboxy group or a sulfo group may adopt a salt structure by a hydrogen atom being dissociated therefrom. In the specific examples below, Et₃NH⁺ represents a triethylammonium cation.

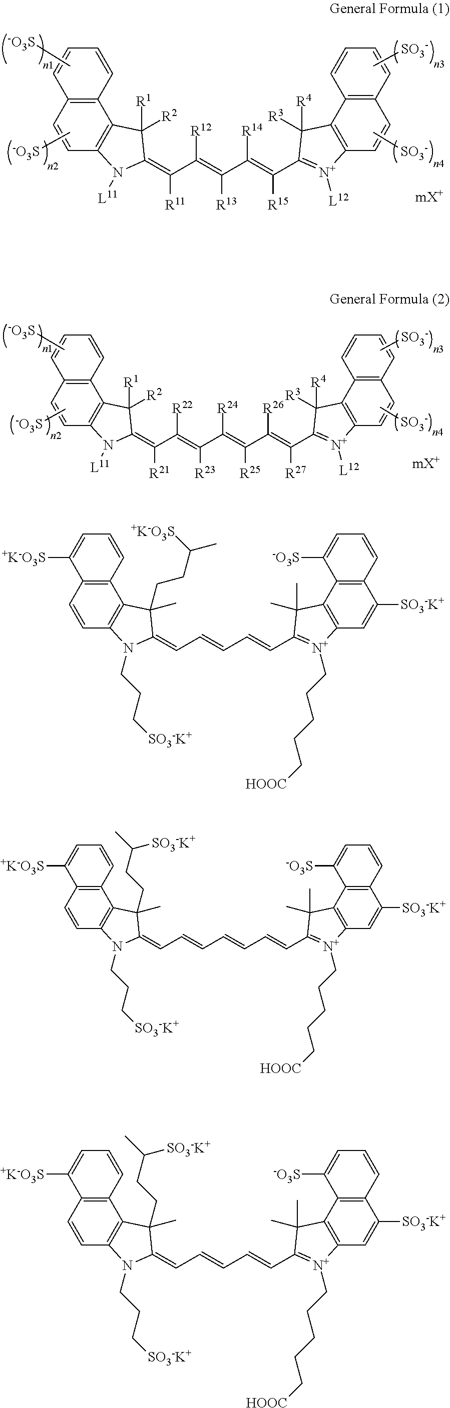

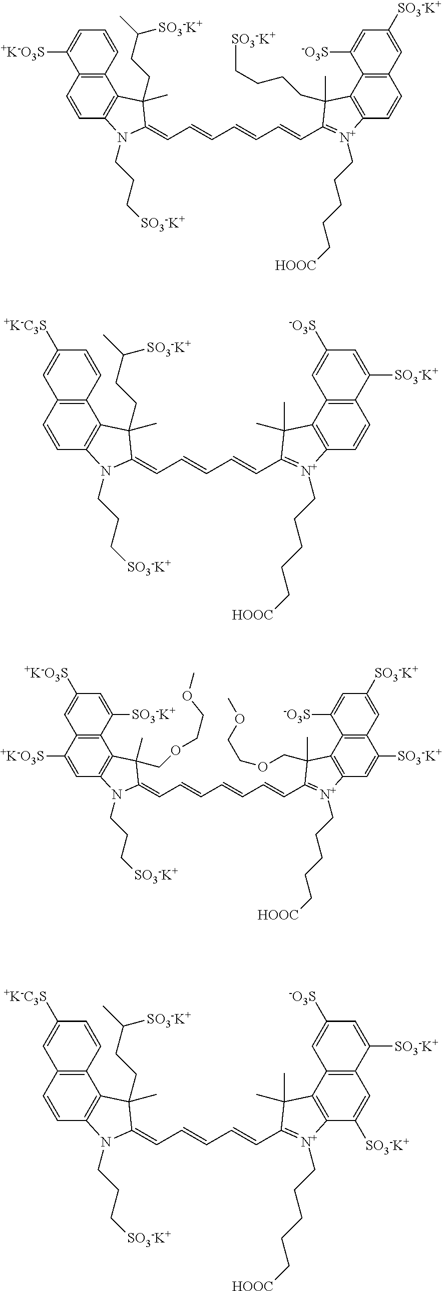

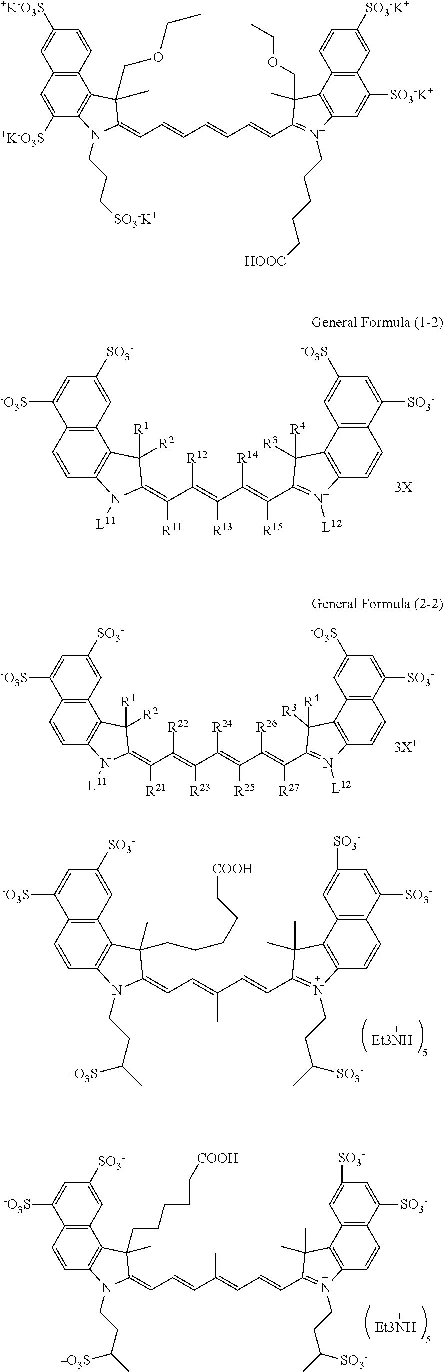

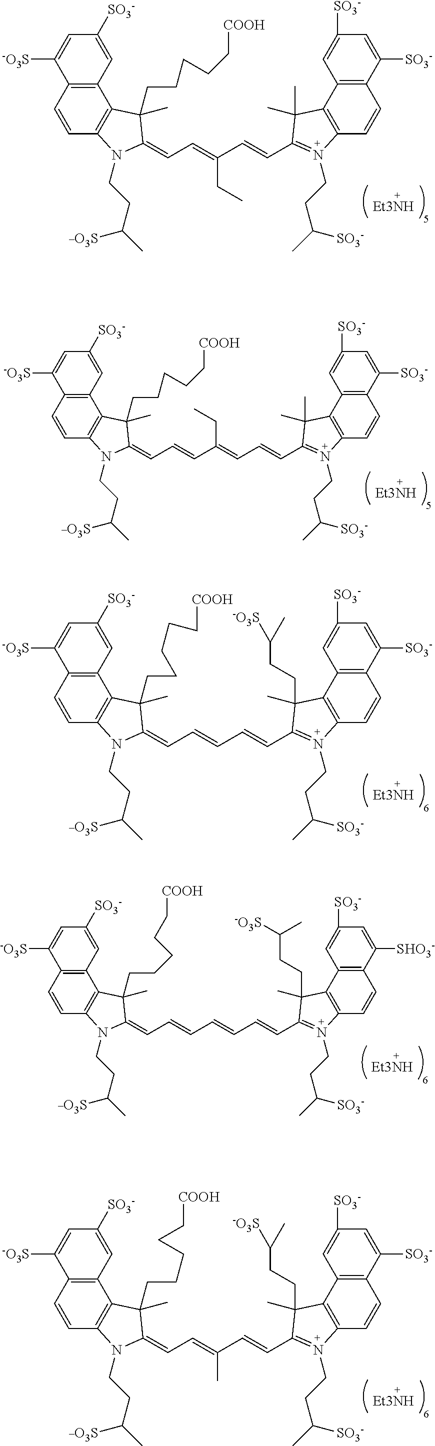

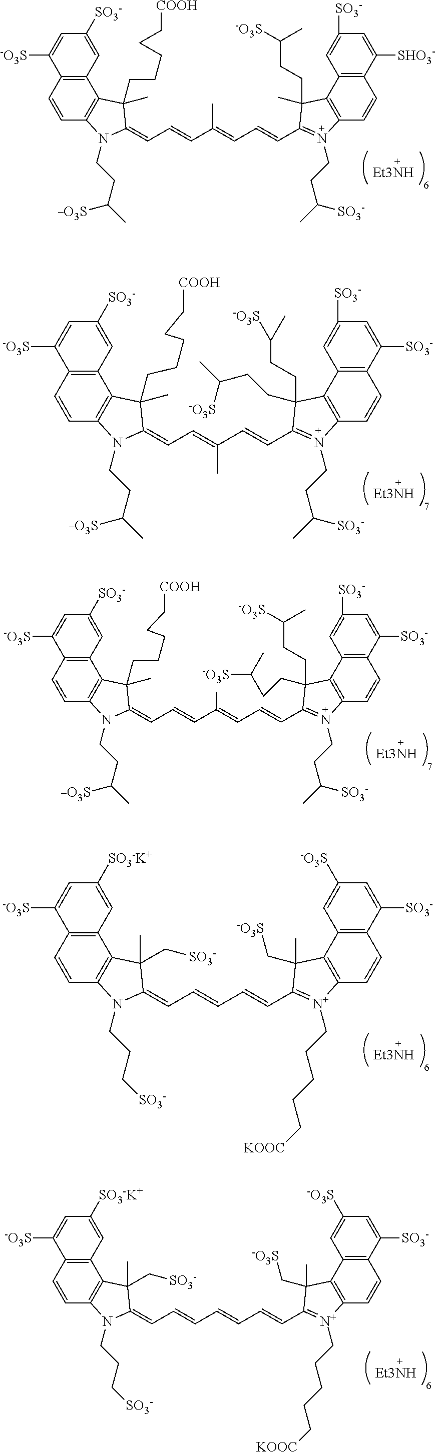

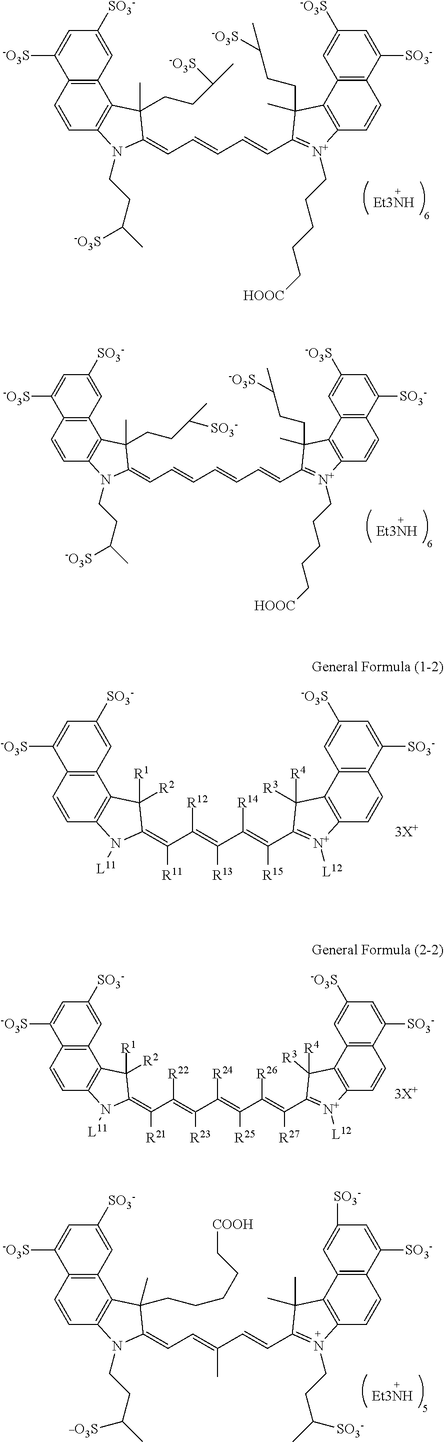

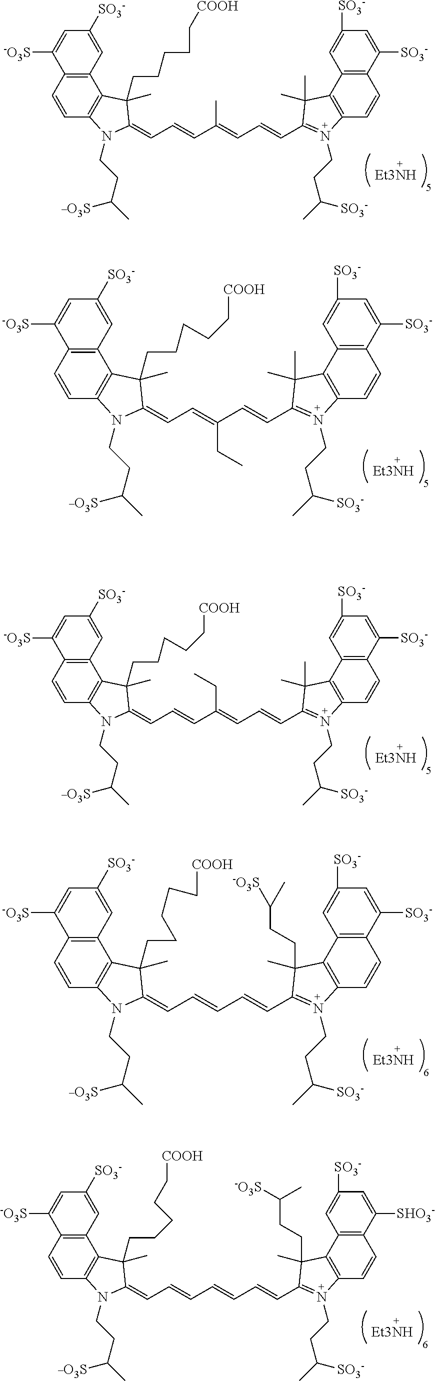

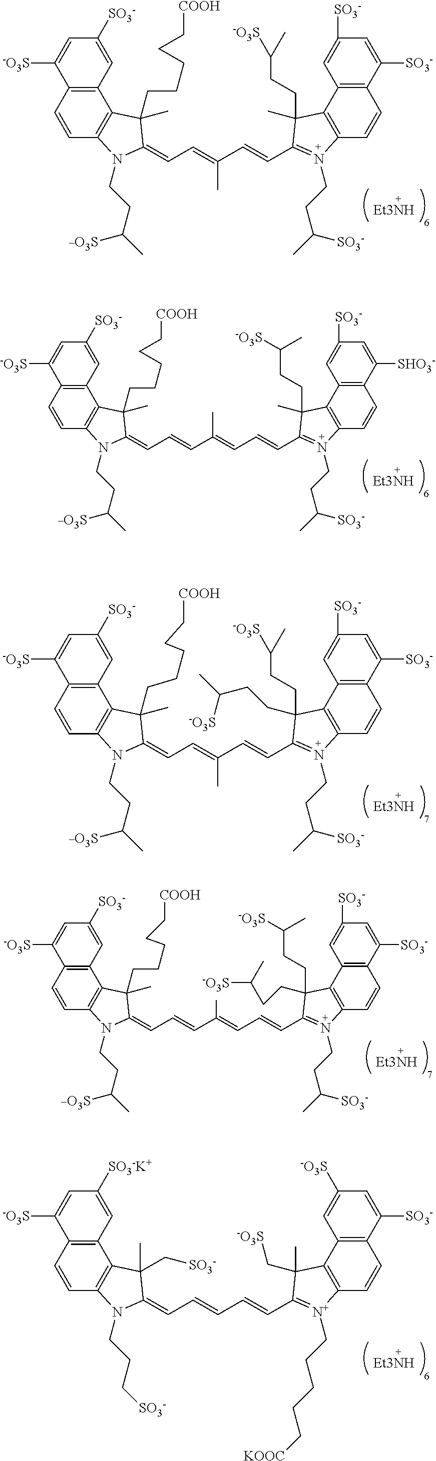

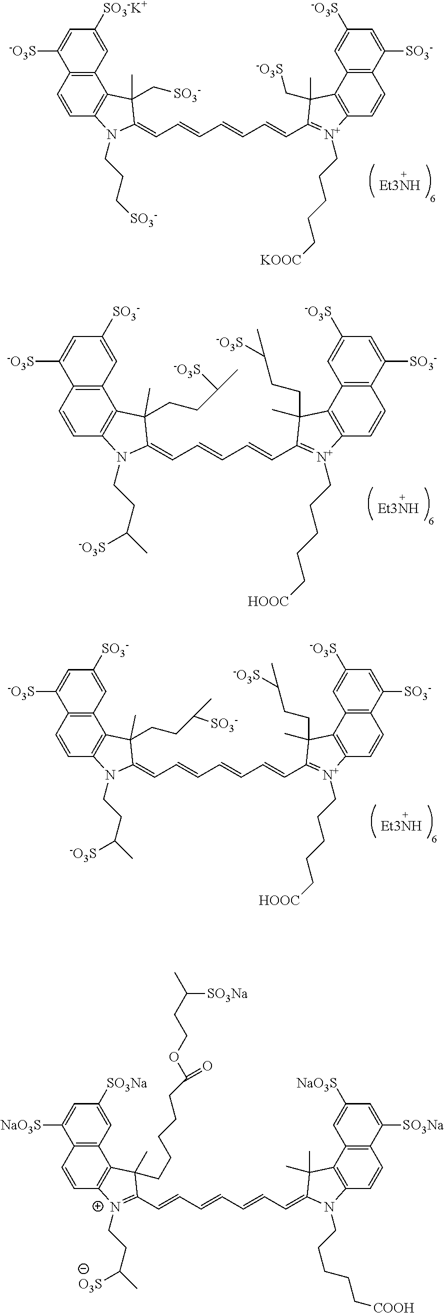

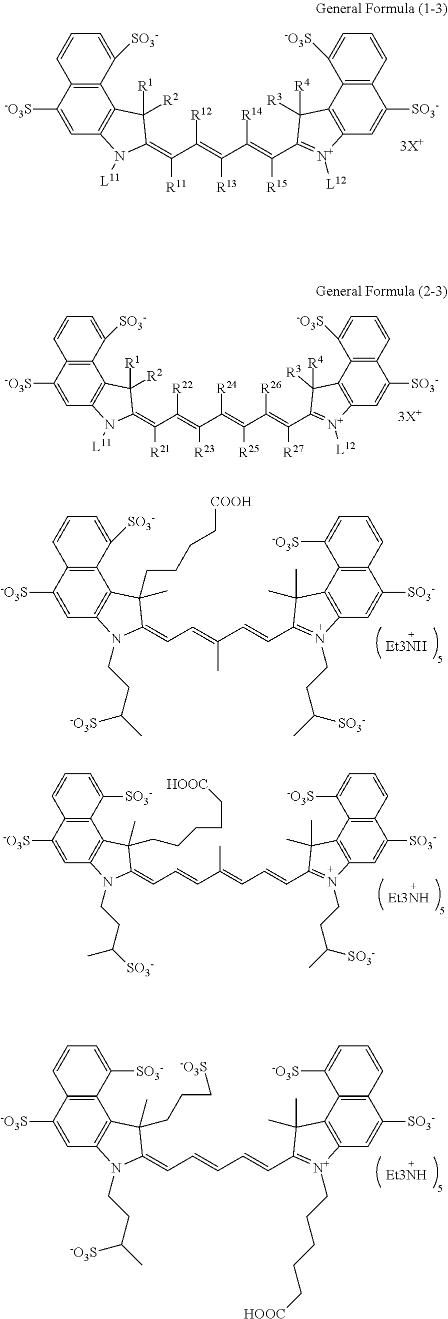

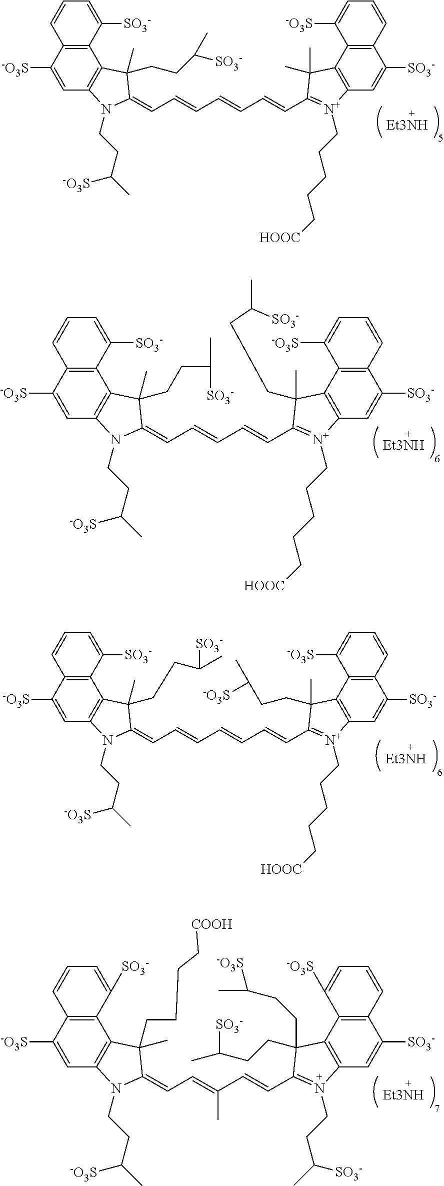

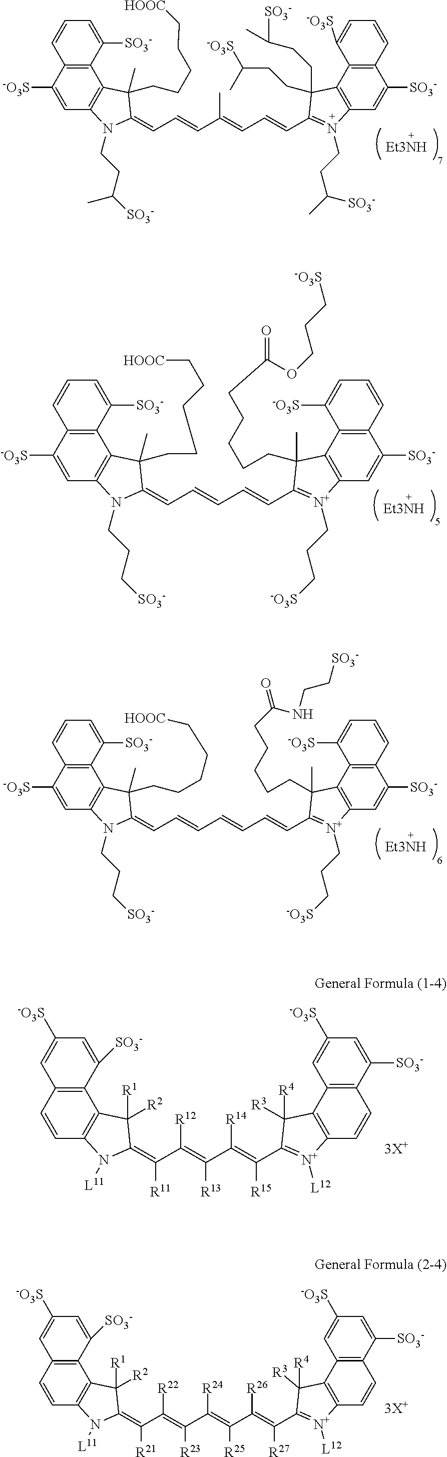

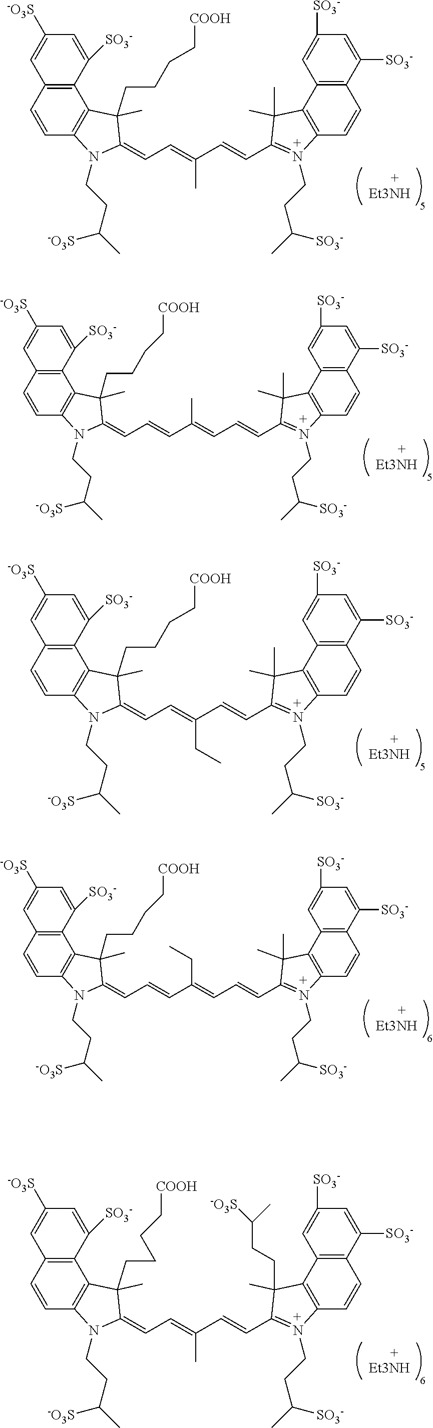

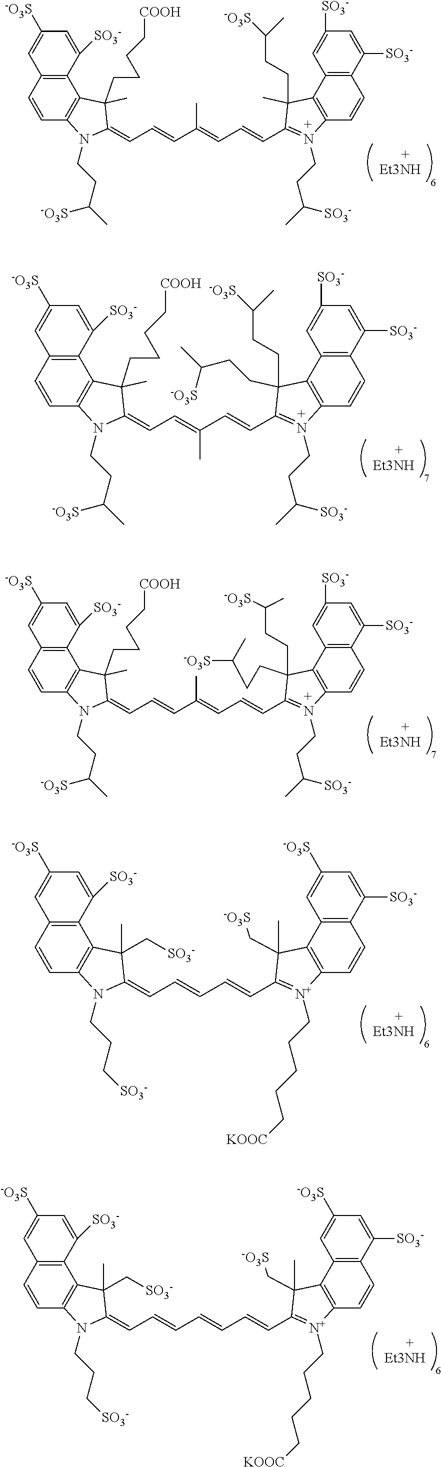

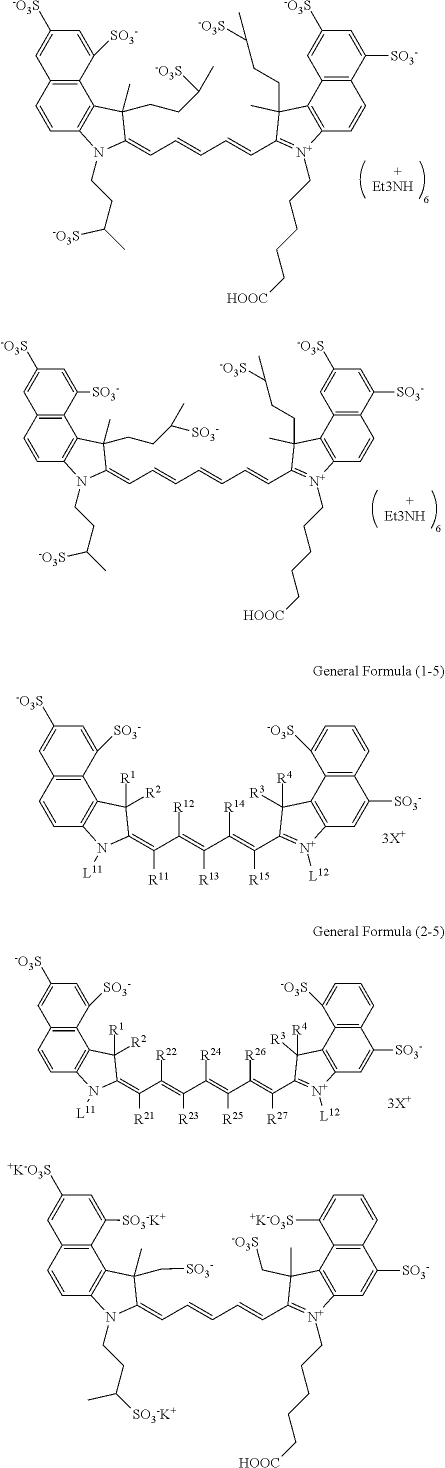

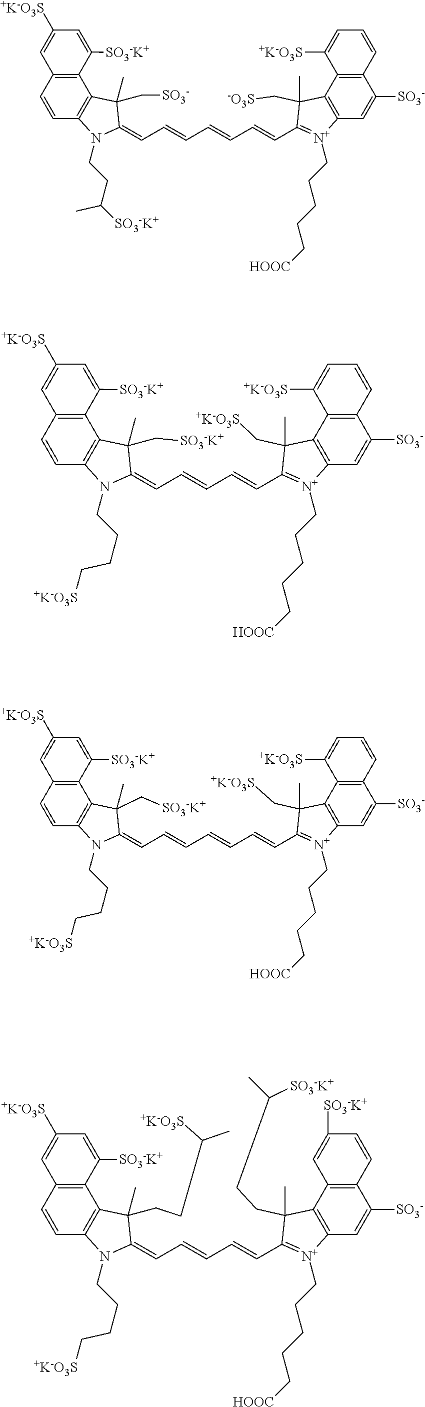

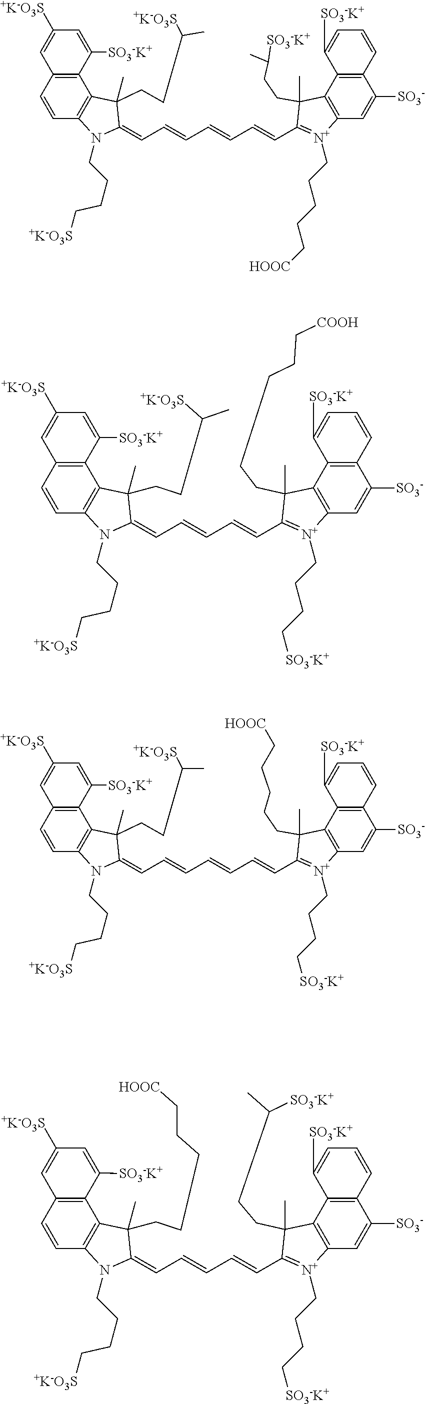

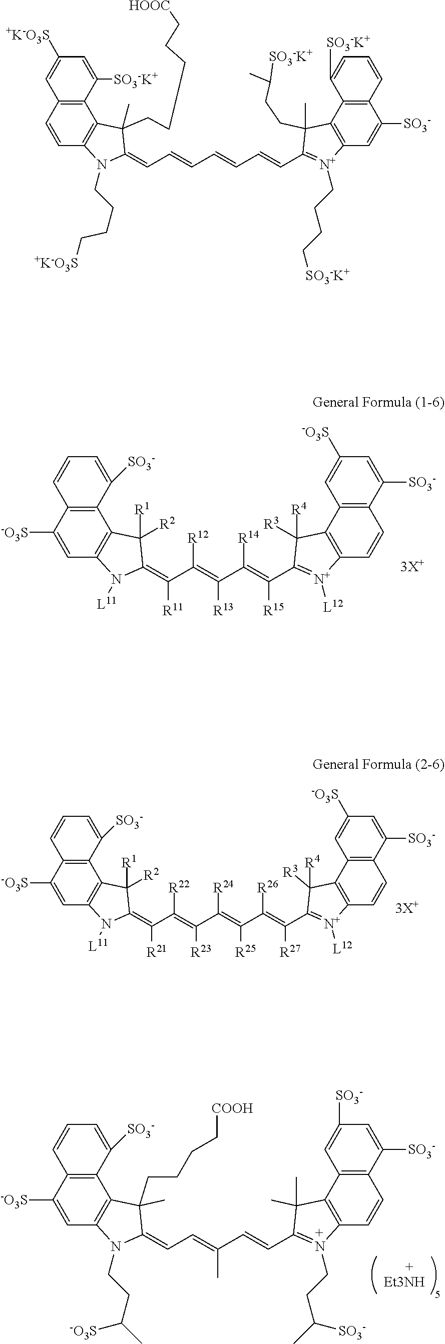

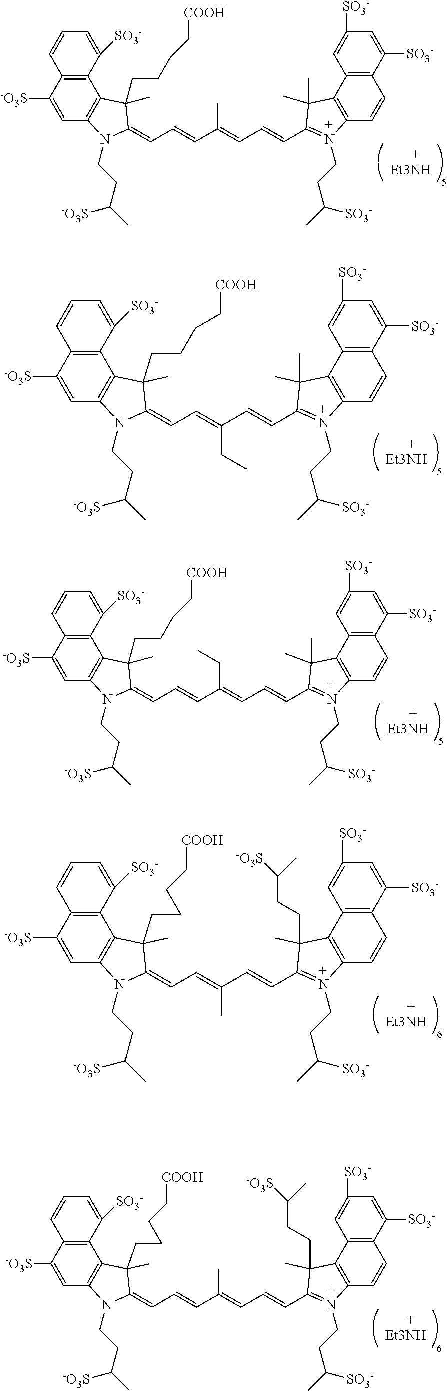

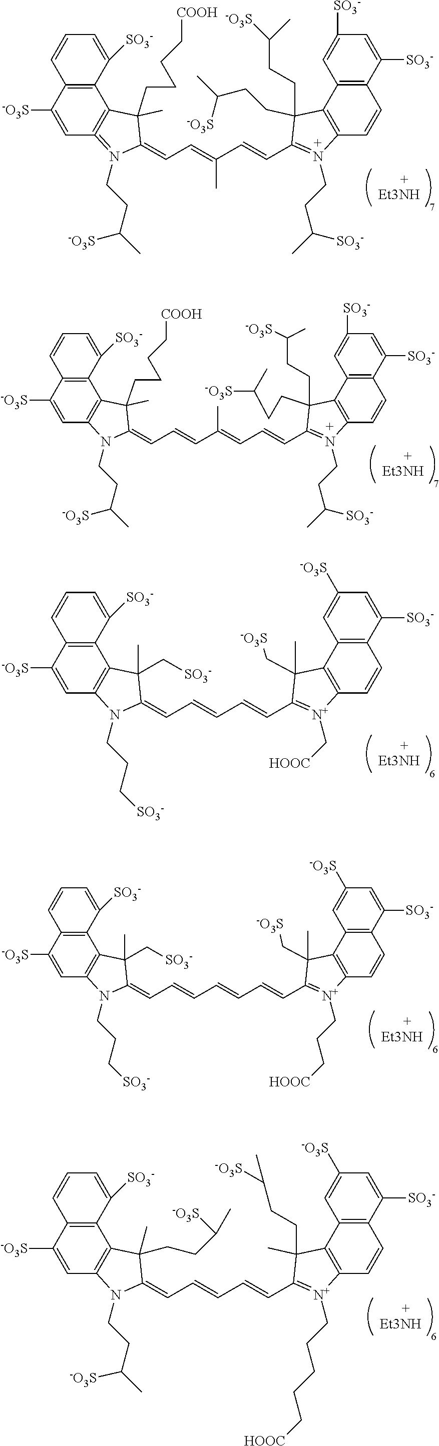

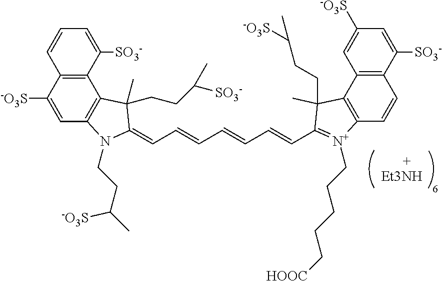

The compound according to the embodiment of the present invention, which is represented by Formula (1) or Formula (2), can be bonded to a biological substance such as a protein, a peptide, an amino acid, a nucleic acid, a sugar chain, or a lipid, with a substituent capable of being bonded to a biological substance contained in at least one of R¹ to R⁴, R¹³, L¹¹, or L¹² in Formula (1) or at least one of R¹ to R⁴, R²⁴, L¹¹ or L¹² in Formula (2), and it can be used as a fluorescently labeled biological substance.

The substituent capable of being bonded to a biological substance can be used without particular limitation as long as it is a group for acting (including adhering) or bonding to a biological substance, and examples thereof include the substituents described in WO2002/026891A and the like. Among them, preferred examples thereof include an NHS ester structure, a maleimide structure, an azido group, an acetylene group, a peptide structure (a polyamino acid structure), a long-chain alkyl group (preferably having 12 to 30 carbon atoms), and a quaternary ammonium group.

Among the compounds according to the embodiment of the present invention, which are represented by Formula (1) or Formula (2), specific examples of the compound in which at least any one of R¹ to R⁴, R¹³, R²⁴, L¹¹ or L¹² has at least a substituent capable of being bonded to a biological substance include an exemplary compound of a fluorescently labeled biological substance described later. Further, specific examples thereof also include a form of an exemplary compound of the compound according to the embodiment of the present invention represented by Formula (1) or Formula (2), in which a substituent capable of being bonded to a biological substance is contained and which is shown as an exemplary compound of the fluorescently labeled biological substance described later. It is noted that the present invention is not limited to these compounds. For example, in the specific examples thereof, a group having a dissociative hydrogen atom such as a specific hydrophilic group Pi may adopt a salt structure by a hydrogen atom being dissociated therefrom.

The compound according to the embodiment of the present invention, which is represented by Formula (1) or Formula (2), can be synthesized by a known method except that the compound structure is the structure regulated by Formula (1) or Formula (2). For example, the methods described in WO2005/044923A, WO2005/044923A, and the like can be mentioned.

A compound having a substituent capable of binding to a biological substance can be synthesized by a known method except that the compound structure is the structure regulated by Formula (1) or Formula (2). For example, Bioconjugate Techniques (Third Edition, written by Greg T. Hermanson) can be referred to.

<<Fluorescently Labeled Biological Substance>>

The fluorescently labeled biological substance according to the embodiment of the present invention is a substance in which the compound according to the embodiment of the present invention, which is represented by Formula (1) or Formula (2), is bonded to a biological substance. Since the compound according to the embodiment of the present invention, which is represented by Formula (1) or Formula (2), has fluorescence and exhibits an absorption wavelength peak suitable for color development in the near infrared range and an excellent fluorescence intensity, it can be preferably used for a fluorescently labeled biological substance. The bond between the compound represented by Formula (1) or (2) and a biological substance may have a configuration in which the compound represented by Formula (1) or (2) and the biological substance are directly bonded or a configuration of being linked through a linking group.

Preferred examples of the biological substance include a protein, a peptide, an amino acid, a nucleic acid, a sugar chain, and a lipid. Preferred examples of the protein include an antibody, and preferred examples of the lipid include a phospholipid, a fatty acid, sterol, where a phospholipid is more preferable.

Among the above biological substances, the clinically useful substance is not particularly limited, but examples thereof include immunoglobulins such as immunoglobulin (Ig) G, IgM, IgE, IgA, and IgD; plasma proteins such as complement, C-reactive protein (CRP), ferritin, α₁ microglobulin, β₂ microglobulin, and antibodies thereof; tumor markers such as α-fetoprotein, carcinoembryonic antigen (CEA), prostate acid phosphatase (PAP), carbohydrate antigen (CA) 19-9, and CA-125, and antibodies thereof; hormones such as luteinizing hormone (LH), follicle-stimulating hormone (FSH), human ciliated gonadotropin (hCG), estrogen, and insulin, and antibodies thereof; and viral infection-related substances of viruses such HIV and ATL, hepatitis B virus (HBV)-related antigens (HBs, HBe, and HBc), human immunodeficiency virus (HIV), adult T-cell leukemia (ATL), and antibodies thereof.

The examples thereof further include bacteria such as Corynebacterium diphtheriae, Clostridium botulinum, mycoplasma, and Treponema pallidum, and antibodies thereof, protozoa such as Toxoplasma, Trichomonas, Leishmania, Trypanosoma, and malaria parasites, and antibodies thereof, embryonic stem (ES) cells such as ELM3, HM1, KH2, v6.5, v17.2, v26.2 (derived from mice, 129, 129/SV, C57BL/6, and BALB/c), and antibodies thereof, antiepileptic drugs such as phenytoin and phenobarbital; cardiovascular drugs such as quinidine and digoxin; anti-asthma drugs such as theophylline; drugs such as antibiotics such as chloramphenicol and gentamicin, and antibodies thereof; and enzymes, extracellular toxins (for example, styrelidine O), and the like, and antibodies thereof. In addition, antibody fragments such as Fab′2, Fab, and Fv can also be used.

Examples of the specific configuration in which the compound according to the embodiment of the present invention, which is represented by Formula (1) or Formula (2), (hereinafter, also abbreviated as the compound (1) or (2)) and the biological substance interact with each other to be bonded include the configurations described below,

i) non-covalent bond (for example, hydrogen bond, ionic bond including chelate formation) or covalent bond between a peptide in the compound (1) or (2) and a peptide in the biological substance,

ii) van der Waals force between a long-chain alkyl group in the compound (1) or (2) and a lipid bilayer, a lipid, or the like in the biological substance,

iii) an amide bond formed by reacting an N-hydroxysuccinimide ester (NHS ester) in the compound (1) or (2) with an amino group in the biological substance,

iv) a thioether bond formed by reacting a maleimide group in the compound (1) or (2) with a sulfanyl group (-SH) in the biological substance, and

v) a formation of a triazole ring, which is formed by Click reaction between an azido group in the compound (1) or (2) and an acetylene group in the biological substance, or the Click reaction between an acetylene group in the compound (1) or (2) and an azido group in the biological substance.

In addition to the forms of the i) to v), the bond can be formed according to another form, for example, which is described in “Lucas C. D. de Rezende and Flavio da Silva Emery, A Review of the Synthetic Strategies for the Development of BODIPY Dyes for Conjugation with Proteins, Orbital: The Electronic Journal of Chemistry, 2013, Vol 5, No.1, p. 62-83”. Further, the method described in the same document can be appropriately referred to for the preparation of the fluorescently labeled biological substance according to the embodiment of the present invention.

Hereinafter, among the compounds according to the embodiment of the present invention, which are represented by Formula (1) or Formula (2), a compound in which at least any one of R¹ to R⁴, R¹³, R^{24,} L¹¹, or L¹² has a substituent capable of being bonded to a biological substance, and specific examples of the fluorescently labeled biological substance according to the embodiment of the present invention, which is obtained from the biological substance that is bonded to the above compound with interaction; however, the present invention is not limited to these compounds. In the following specific examples, a group having a dissociative hydrogen atom such as a sulfo group may adopt a salt structure by a hydrogen atom being dissociated therefrom. Et represents an ethyl group.

| Compound Example | Product (bonding mode) |
|---|---|
| 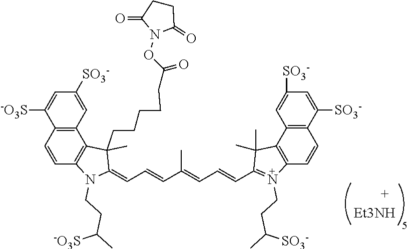 NHS ester structure | 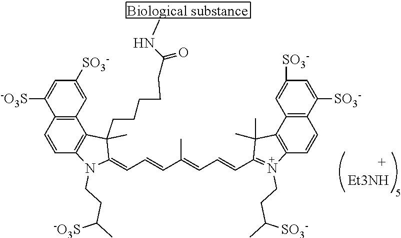 Bonding through amino group of biological substance |
| 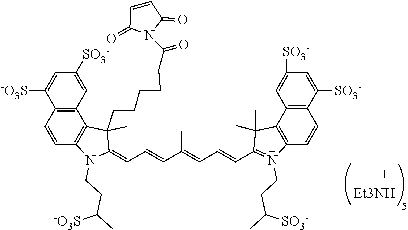 Maleimide structure | 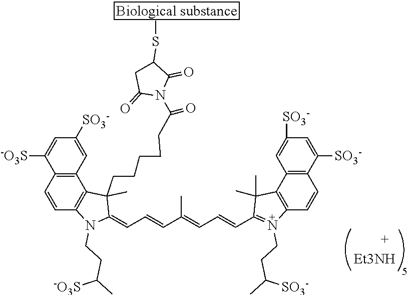 |
| | Bonding through sulfanyl group of biological substance |
| 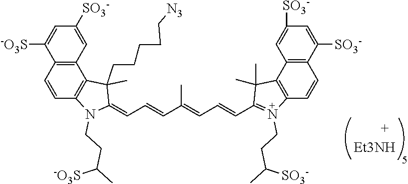 Azide structure | 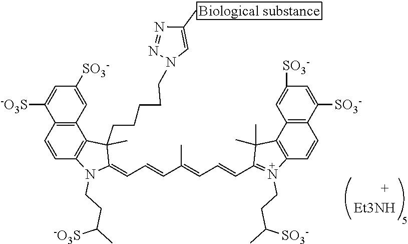 |
| | Click reaction through acetylene group of biological substance |
| 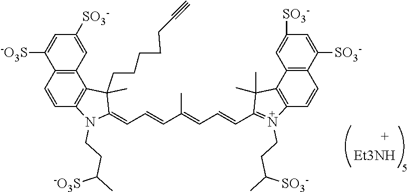 Acetylene structure | 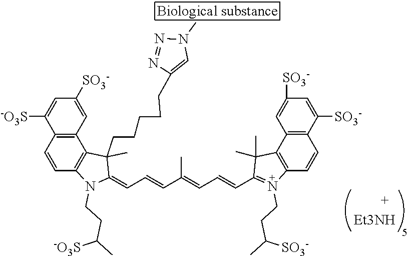 |
| | Click reaction through azide group of biological substance |
| 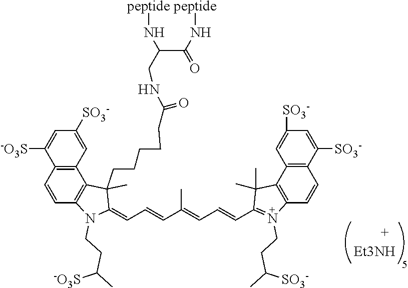 Polypeptide structure (polyamino acid structure) | 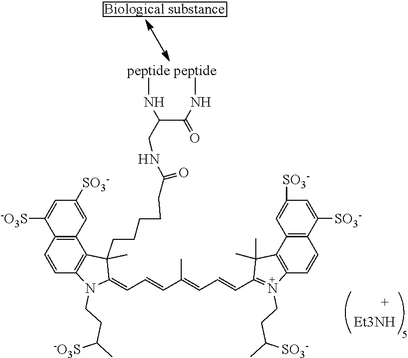 |
| | Bonding through polypeptide of biological substance |
| 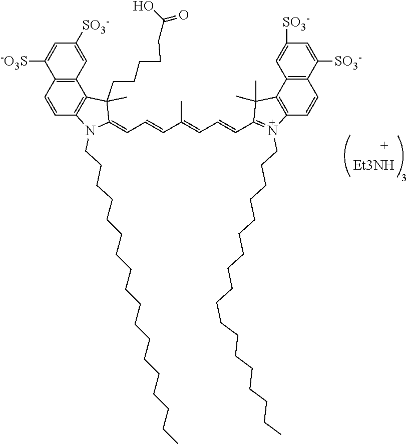 | Van der Waals force through lipid bilayer, phospholipid, or the like of biological substance |
| Long-chain alkyl structure | |

<Reagent Containing Fluorescently Labeled Biological Substance>

In the reagent containing the fluorescently labeled biological substance according to the embodiment of the present invention, the form of the fluorescently labeled biological substance according to the embodiment of the present invention, for example, a solution form dissolved in an aqueous medium such as physiological saline and a phosphate buffer solution, and a solid form such as a fine particle powder or a lyophilized powder, is not particularly limited and can be appropriately selected depending on the purpose of use.

For example, in a case where the fluorescently labeled biological substance according to the embodiment of the present invention is used as a fluorescence labelling reagent, it can be used as a reagent containing the fluorescently labeled biological substance having any one of the forms described above.

<Use Application of Fluorescently Labeled Biological Substance>

The fluorescently labeled biological substance according to the embodiment of the present invention, obtained from the compound according to the embodiment of the present invention, which is represented by Formula (1) or Formula (2), can exhibit an excellent fluorescence intensity and stably detect fluorescence emitted from the fluorescently labeled biological substance excited by light irradiation. As a result, the fluorescently labeled biological substance according to the embodiment of the present invention can be applied to various techniques using the fluorescence labelling, and it can be suitably used, for example, as a fluorescence labeling reagent in a multicolor WB and a reagent for in vivo fluorescence imaging.

The fluorescence detection carried out using the fluorescently labeled biological substance according to the embodiment of the present invention usually includes the following processes (i) to (iii) or (iv) to (vii). The fluorescence detection including the processes (i) to (iii) corresponds to the direct method using a primary antibody fluorescently labeled with the compound according to the embodiment of the present invention, and the fluorescence detection including the processes (iv) to (vii) corresponds to the indirect method using a secondary antibody fluorescently labeled with the compound according to the embodiment of the present invention.

(i) The process of preparing each of the following (a) and (b)

(a) A sample containing a targeted biological substance (hereinafter, also referred to as a “target biological substance”)

(b) A fluorescently labeled biological substance according to the embodiment of the present invention (hereinafter, also referred to as a “fluorescently labeled biological substance A according to the embodiment of the present invention”) obtained by bonding the biological substance (hereinafter, also referred to as a “primary biological substance”) capable of binding to the target biological substance in the above (a) to the compound according to the embodiment of the present invention

(ii) The process of preparing a conjugate (hereinafter, also referred to as a “fluorescently labeled conjugate A”) in which the target biological substance in the above (a) is bonded to the primary biological substance in the fluorescently labeled biological substance A according to the embodiment of the present invention in the above (b)

(iii) The process of irradiating the fluorescently labeled conjugate A with light having the range of the wavelength which is absorbed by the fluorescently labeled biological substance A according to the embodiment of the present invention, and detecting the fluorescence emitted by the fluorescently labeled biological substance A according to the embodiment of the present invention

(iv) The process of preparing each of the following (c) to (e)

(c) A sample containing a target biological substance

(d) A biological substance capable of binding to the target biological substance in the above (c) (hereinafter, also referred to as a “primary biological substance”)

(e) A fluorescently labeled biological substance according to the embodiment of the present invention (hereinafter, also referred to as a “fluorescently labeled biological substance B according to the embodiment of the present invention”) obtained by bonding the biological substance (hereinafter, also referred to as a “secondary biological substance”) capable of binding to the primary biological substance in the above (d) to the compound according to the embodiment of the present invention (hereinafter, also referred to as a “fluorescently labeled biological substance B according to the embodiment of the present invention”)

(v) The process of preparing a conjugate (hereinafter, also referred to as a “conjugate b”) in which the target biological substance in the above (c) is bonded to the primary biological substance of the above (d)

(vi) The process of preparing a conjugate (hereinafter, also referred to as a “fluorescently labeled conjugate B2”) in which the primary biological substance in the conjugate (b) is bonded to the secondary biological substance in the fluorescently labeled biological substance B according to the embodiment of the present invention

(vii) The process of irradiating the fluorescently labeled conjugate B2 with light having the range of the wavelength which is absorbed by the fluorescently labeled biological substance B according to the embodiment of the present invention, and detecting the fluorescence emitted by the fluorescently labeled biological substance B according to the embodiment of the present invention

Examples of the biological substance (the primary biological substance) capable of binding to the target biological substance and the biological substance (the secondary biological substance) capable of binding to the primary biological substance include the biological substances in the fluorescently labeled biological substance according to the embodiment of the present invention. The above biological substance can be appropriately selected in accordance with the target biological substance (a biological substance in the test object) or the primary biological substance, and a biological substance capable of specifically binding to the biological substance in the test object or to the primary biological substance can be selected.

Examples of the protein among the target biological substances include a protein, which is a so-called disease marker. The disease marker is not particularly limited, and examples thereof include α-fetoprotein (AFP), protein induced by vitamin K absence or antagonist II (PIVKA-II), breast carcinoma-associated antigen (BCA) 225, basic fetoprotein (BFP), carbohydrate antigen (CA) 15-3, CA19-9, CA72-4, CA125, CA130, CA602, CA54/61 (CA546), carcinoembryonic antigen (CEA), DUPAN-2, elastase 1, immunosuppressive acidic protein (TAP), NCC-ST-439, γ-seminoprotein (γ-Sm), prostate specific antigen (PSA), prostatic acid phosphatase (PAP), nerve specific enolase (NSE), Iba1, amyloid β, tau, flotillin, squamous cell carcinoma associated antigen (SCC antigen), sialyl LeX-i antigen (SLX), SPan-1, tissue polypeptide antigen (TPA), serial Tn antigen (STN), cytokeratin (CYFRA) pepsinogen (PG), C-reactive protein (CRP), serum amyloid A protein (SAA), myoglobin, creatine kinase (CK), troponin T, and ventricular muscle myosin light chain I.

The target biological substance may be a bacterium. Examples of the bacterium include a bacterium to be subjected to a cellular and microbiological test, which is not particularly limited. Specific examples thereof include Escherichia coli, Salmonella, Legionella, and bacteria causing problems in public health.

The target biological substance may be a virus antigen. Although the virus antigen is not particularly limited, examples of the virus antigen include hepatitis virus antigens such as hepatitis C and B virus antigens, p24 protein antigen of HIV virus, and pp65 protein antigen of cytomegalovirus (CMV), and E6 and E7 protein antigens of human papillomavirus (HPV).

In the above (i) or (iv), the sample containing the target biological substance is not particularly limited and can be prepared according to a conventional method.

In addition, the fluorescently labeled biological substance according to the embodiment of the present invention is not particularly limited and can be prepared by bonding a biological substance capable of binding to a target biological substance to the compound according to the embodiment of the present invention, according to a conventional method. The form of the bond and the reaction to form the bond are as described above in the fluorescently labeled biological substance according to the embodiment of the present invention.

In the above (v), the fluorescently labeled biological substance may be directly bonded to the primary biological substance or may be bonded through another biological substance which is different from the target biological substance and the primary biological substance. Further, in the above (vi), the primary biological substance in the conjugate b may be directly bonded to the secondary biological substance in the fluorescently labeled biological substance B according to the embodiment of the present invention or may be bonded through another biological substance which is different from the primary biological substance and the secondary biological substance.

The fluorescently labeled biological substance according to the embodiment of the present invention can be used as a fluorescently labeled antibody in both the direct method and the indirect method but is preferably used as a fluorescently labeled antibody in the indirect method.

In the above (ii) or (v) and the (vi), the binding of the fluorescently labeled biological substance or the like according to the embodiment of the present invention to the target biological substance is not particularly limited and can be carried out according to a conventional method.

In the above (iii) or (vii), the wavelength for exciting the fluorescently labeled biological substance according to the embodiment of the present invention is not particularly limited as long as the wavelength is an emission wavelength capable of exciting the fluorescently labeled biological substance according to the embodiment of the present invention.

Since the fluorescently labeled biological substance using the compound (1) has an absorption maximum wavelength in the vicinity of 685 nm (660 to 720 nm), the range of the wavelength of light to be emitted is preferably 630 to 750 nm and more preferably 650 to 730 nm. The fluorescently labeled biological substance using the compound (1) can be suitably used as a fluorescently labeled biological substance that exhibits an excellent fluorescence intensity with respect to a light source for excitation wavelength in the vicinity of 700 nm in the near infrared range of the multicolor WB.

Since the fluorescently labeled biological substance using the compound (2) has an absorption maximum wavelength in the vicinity of 785 nm (760 to 820 nm), the range of the wavelength of light to be emitted is preferably 730 to 850 nm and more preferably 750 to 830 nm. The fluorescently labeled biological substance using the compound (2) can be suitably used as a fluorescently labeled biological substance that exhibits an excellent fluorescence intensity with respect to a light source for excitation wavelength in the vicinity of 800 nm in the near infrared range of the multicolor WB.

The fluorescence excitation light source used in the present invention is not particularly limited as long as it emits an emission having wavelength capable of exciting the fluorescently labeled biological substance according to the embodiment of the present invention, and for example, various laser light sources can be used. In addition, various optical filters can be used to obtain a preferred excitation wavelength or detect only fluorescence.

Other matters in the above (i) to (vii) are not particularly limited, and conditions of a method, a reagent, a device, and the like, which are generally used in the fluorescence detection using fluorescence labelling, can be appropriately selected.

Further, regarding the processes other than the above (i) to (vii) as well, conditions of a method, a reagent, a device, and the like, which are generally used, can be appropriately selected in accordance with various methods using fluorescence labelling.

For example, in the multicolor WB using the fluorescently labeled biological substance according to the embodiment of the present invention, it is possible to detect a target biological substance with excellent fluorescence intensity by preparing a blotted membrane according to a method generally used for a target biological substance (protein separation by electrophoresis, blotting to a membrane, and blocking of a membrane) and using the fluorescently labeled biological substance according to the embodiment of the present invention as a labeled antibody (preferably, as a secondary antibody).

Substituent Group T

In the present invention, the preferred substituents include those selected from the following substituent group T.

In addition, in the present specification, in a case where it is simply described as a substituent, the substituent refers to the substituent group T, and in a case where an individual group, for example, an alkyl group is only described, a corresponding group in the substituent group T is preferably applied.

Further, in the present specification, in a case where an alkyl group is described separately from a cyclic (cyclo)alkyl group, the alkyl group is used to include a linear alkyl group and a branched alkyl group. On the other hand, in a case where an alkyl group is not described separately from a cyclic alkyl group, and unless otherwise specified, the alkyl group is used to include a linear alkyl group, a branched alkyl group, and a cycloalkyl group. This also applies to groups (alkoxy group, alkylthio group, alkenyloxy group, and the like) containing a group capable of having a cyclic structure (alkyl group, alkenyl group, alkynyl group, and the like) and compounds containing a group capable of having a cyclic structure. In a case where a group is capable of forming a cyclic skeleton, the lower limit of the number of atoms of the group forming the cyclic skeleton is 3 or more and preferably 5 or more, regardless of the lower limit of the number of atoms specifically described below for the group that can adopt this structure,

In the following description of the substituent group T, a group having a linear or branched structure and a group having a cyclic structure, such as an alkyl group and a cycloalkyl group, are sometimes described separately for clarity.

As the groups included in the substituent group T, the following groups are included.

An alkyl group (preferably having 1 to 30 carbon atoms, more preferably 1 to 20 carbon atoms, still more preferably 1 to 12 carbon atoms, still more preferably 1 to 8 carbon atoms, still more preferably 1 to 6 carbon atoms, and particularly preferably 1 to 3 carbon atoms), an alkenyl group (preferably having 2 to 30 carbon atoms, more preferably 2 to 20 carbon atoms, still more preferably 2 to 12 carbon atoms, still more preferably 2 to 6 carbon atoms, and even still more preferably 2 to 4 carbon atoms), an alkynyl group (preferably having 2 to 30 carbon atoms, still more preferably 2 to 20 carbon atoms, still more preferably 2 to 12 carbon atoms, still more preferably 2 to 6 carbon atoms, and even still more preferably 2 to 4 carbon atoms), a cycloalkyl group (preferably having 3 to 20 carbon atoms), a cycloalkenyl group (preferably having 5 to 20 carbon atoms), an aryl group (it may be a monocyclic group or may be a condensed ring group (preferably a condensed group in which 2 to 6 rings are condensed); in a case of a condensed ring group, it consists of a 5-membered to 7-membered ring; and the aryl group preferably has 6 to 40 carbon atoms, more preferably 6 to 30 carbon atoms, still more preferably 6 to 26 carbon atoms, and particularly preferably 6 to 10 carbon atoms), a heterocycle group (it has, as a ring-constituting atom, at least one nitrogen atom, an oxygen atom, a sulfur atom, a phosphorus atom, a silicon atom, or selenium atom, may be a monocyclic ring, or may be a condensed ring group (preferably a condensed group in which 2 to 6 rings are condensed); in a case of a monocyclic group, the monocyclic ring is preferably a 5-membered to 7-membered ring and more preferably a 5-membered or 6-membered ring; the heterocycle group preferably has 2 to 40 carbon atoms and more preferably 2 to 20 carbon atoms; and the heterocyclic group includes an aromatic heterocyclic group (a heteroaryl group) and an aliphatic heterocyclic group (an aliphatic heterocyclic group), an alkoxy group (preferably having 1 to 20 carbon atoms, and more preferably having 1 to 12 carbon atoms), an alkenyloxy group (preferably having 2 to 20 carbon atoms, and more preferably having 2 to 12 carbon atoms), and an alkynyloxy group (preferably having 2 to 20 carbon atoms, and more preferably having 2 to 12 carbon atoms), a cycloalkyloxy group (preferably having 3 to 20 carbon atoms), an aryloxy group (preferably having 6 to 40 carbon atoms, more preferably having 6 to 26 carbon atoms, and still more preferably having 6 to 14 carbon atoms), a heterocyclic oxy group (preferably having 2 to 20 carbon atoms),

an alkoxycarbonyl group (preferably having 2 to 20 carbon atoms), a cycloalkoxycarbonyl group (preferably having 4 to 20 carbon atoms), an aryloxycarbonyl group (preferably having 6 to 20 carbon atoms), an amino group (preferably having 0 to 20 carbon atoms; the amino group includes an unsubstituted amino group (—NH₂), a (mono- or di-) alkylamino group, a (mono- or di-) alkenylamino group, a (mono- or di-) alkynylamino group, a (mono- or di-) cycloalkylamino group, a (mono- or di-) cycloalkenylamino group, a (mono- or di-) arylamino group, or a (mono- or di-) heterocyclic amino group, where each of the above groups substituting an unsubstituted amino group has the same definition as the corresponding group in the substituent group T), a sulfamoyl group (preferably having 0 to 20 carbon atoms; the sulfamoyl group is preferably an alkyl, cycloalkyl, or aryl sulfamoyl group), an acyl group (preferably having 1 to 20 carbon atoms, and more preferably having 2 to 15 carbon atoms), an acyloxy group (preferably having 1 to 20 carbon atoms), a carbamoyl group (preferably having 1 to 20 carbon atoms; the carbamoyl group is preferably an alkyl, cycloalkyl, or aryl carbamoyl group),

an acylamino group (preferably having 1 to 20 carbon atoms), a sulfonamide group (preferably having 0 to 20 carbon atoms and preferably an alkyl, cycloalkyl, or aryl sulfonamide group), an alkylthio group (preferably having 1 to 20 carbon atoms and more preferably 1 to 12 carbon atoms), a cycloalkylthio group (preferably having 3 to 20 carbon atoms), an arylthio group (preferably having 6 to 40 carbon atoms, more preferably 6 to 26 carbon atoms, and still more preferably 6 to 14 carbon atoms), a heterocyclic thio group (preferably having 2 to 20 carbon atoms), an alkyl, cycloalkyl, or aryl sulfonyl group (preferably having 1 to 20 carbon atoms),

a silyl group (preferably having 1 to 30 carbon atoms and more preferably 1 to 20 carbon atoms, and preferably substituted with an alkyl, aryl, alkoxy, or aryloxy), a silyloxy group (preferably having 1 to 20 carbon atoms and preferably substituted with an alkyl, aryl, alkoxy, or aryloxy), a hydroxy group, a cyano group, a nitro group, a halogen atom (for example, a fluorine atom, a chlorine atom, a bromine atom or an iodine atom), an oxygen atom (specifically replacing >CH₂ which constitutes a ring with >C═O), a carboxy group (—CO₂H), a phosphono group [—PO(OH)₂], a phosphonooxy group [—O—PO(OH)₂], a sulfo group (—SO₃H), a boric acid group [—B(OH)₂], an onio group (an ammonio group including a cyclic ammonio group, which contains a sulfonio group(—SH₂⁺) or a phosphonio group (—PH3⁺), and preferably has 0 to 30 carbon atoms and more preferably 1 to 20 carbon atoms), a sulfanyl group (—SH), an amino acid residue, or a polyamino acid residue.

Further, the substituent group T includes a carboxy group, a phosphono group, a sulfo group, an onio group, an amino acid residue, or the above-described alkyl group, alkenyl group, alkynyl group, cycloalkyl group, cycloalkenyl group, aryl group, heterocycle group, alkoxy group, alkenyloxy group, alkynyloxy group, cycloalkyloxy group, aryloxy group, heterocyclic oxy group, alkoxycarbonyl group, cycloalkoxycarbonyl group, aryloxycarbonyl group, amino group, sulfamoyl group, acyl group, acyloxy group, carbamoyl group, acylamino group, sulfonamide group, alkylthio group, cycloalkylthio group, arylthio group, heterocyclic thio group, and an alkyl, cycloalkyl, or aryl sulfonyl group, where this above-described group has a polyamino acid residue as a substituent.

The substituent selected from the substituent group T is more preferably an alkyl group, an alkenyl group, a cycloalkyl group, an aryl group, a heterocycle group, an alkoxy group, a cycloalkoxy group, an aryloxy group, an alkoxycarbonyl group, a cycloalkoxycarbonyl group, an amino group, an acylamino group, a cyano group or a halogen atom, and particularly preferably an alkyl group, an alkenyl group, an aryl group, a heterocycle group, an alkoxy group, an alkoxycarbonyl group, an amino group, an acylamino group, or a cyano group.

The substituent selected from the substituent group T also includes a group obtained by combining a plurality of the above groups, unless otherwise specified. For example, in a case where a compound, a substituent, or the like contains an alkyl group, an alkenyl group, or the like, the alkyl group, the alkenyl group, or the like may be substituted or unsubstituted. In addition, in a case where a compound, a substituent, or the like contains an aryl group, a heterocycle group, or the like, the aryl group, the heterocycle group, or the like may be a monocyclic ring or a fused ring moiety, and may be substituted or unsubstituted.

### EXAMPLES

Hereinafter, the present invention will be described in more detail based on Examples, but the present invention is not limited thereto.

Compounds (1) to (6) and comparative compounds (1) and (3) used in Examples are shown below.

It is noted that in the compounds of Examples, the sulfo group and the carboxy group may include a salt structure (for example, a potassium salt, a sodium salt, or an N,N-diisopropylethylamine (DIPEA) salt), even unless otherwise specified. Et represents an ethyl group.

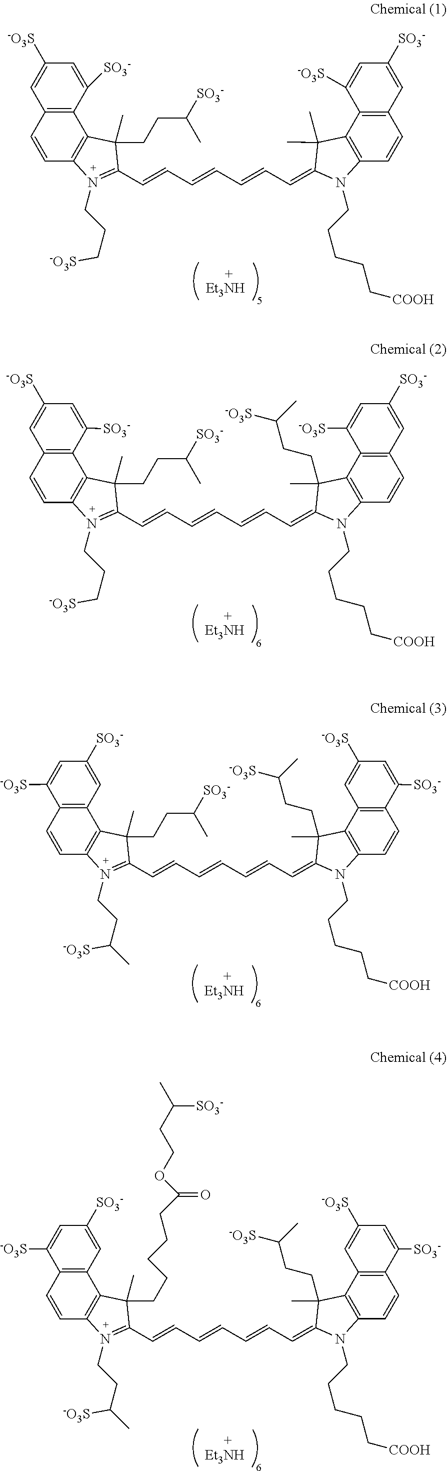

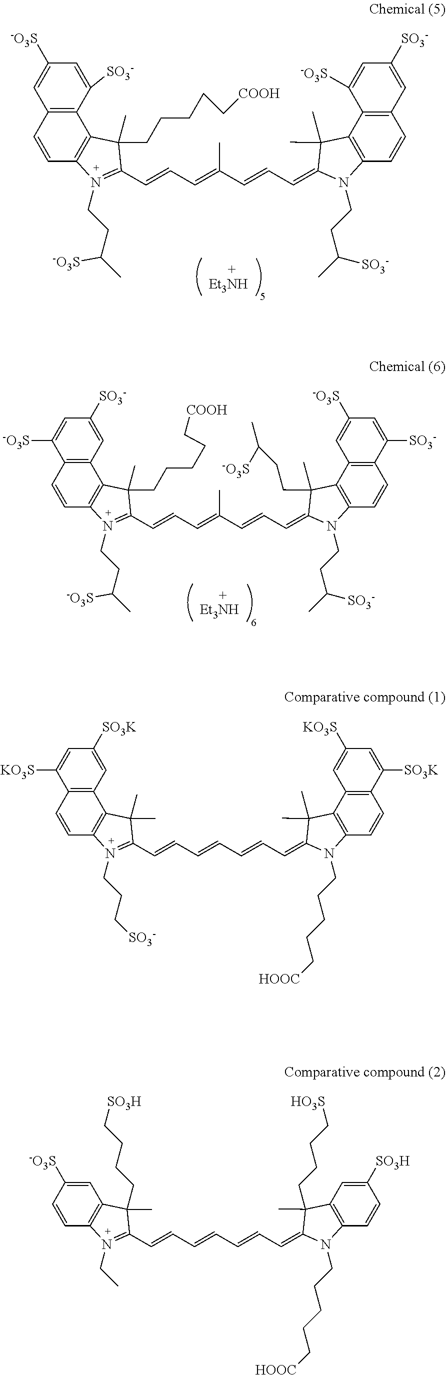

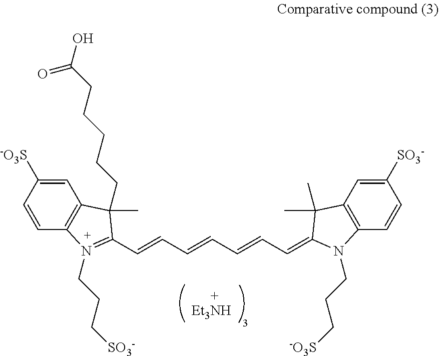

The comparative compound (1) is the compound of Formula (8) described in JP2010-195764A.

The comparative compound (2) is the compound (3) described in WO2005/044923A. The comparative compound (3) is the compound (21) described in WO2002/026891A.

The comparative compounds (1) to (3) were synthesized according to the methods described in the respective documents.

In addition, comparative labeled antibodies (1) to (3) were synthesized according to the same method as the method of synthesizing the labeled antibody (1) described later.

The methods of synthesizing the compounds (1) to (6) and labeled antibodies matching with these compounds, which are used in each Example, are described in detail below, but the starting materials, the dye intermediates, and the synthetic routes are not limited thereto.

In the following synthetic route, room temperature means 25° C.

Unless otherwise specified, SNAP Ultra C18 (manufactured by Biotage, LLC) or Sfar C18 (manufactured by Biotage, LLC) was used as the carrier in the reverse phase column chromatography. The mixing ratio in the eluent is based on a volume ratio. For example, “acetonitrile:water=from 0:100 to 20:80” means that the eluent of “acetonitrile:water=0:100” was changed to an eluent of “acetonitrile:water=20:80”.

For the preparative high performance liquid chromatography (HPLC), 2767 (product name, manufactured by Waters Corporation)] was used.

The MS spectrum was measured by ACQUITY SQD LC/MS System [manufactured by Waters Corporation, ionization method: electrospray Ionization (ESI)] or LCMS-2010EV [manufactured by Shimadzu Corporation, ionization method: an ionization method simultaneously carrying out ESI and atmospheric pressure chemical ionization (APCI)].

Unless otherwise specified, MS (ESI m/z): [M+H⁺]⁺ means a value obtained by removing all Et₃NH⁺, which is a counter cation, from the compound and adding H⁺so that the charge as the compound becomes +1, and MS (ESI m/z):[M−H⁺]⁻ means a value obtained by removing all Et₃NH⁺, which is a counter cation, from the compound and removing H⁺ so that the charge as the compound becomes −1.

### Synthesis Example 1

A compound (1) was synthesized based on the following scheme.

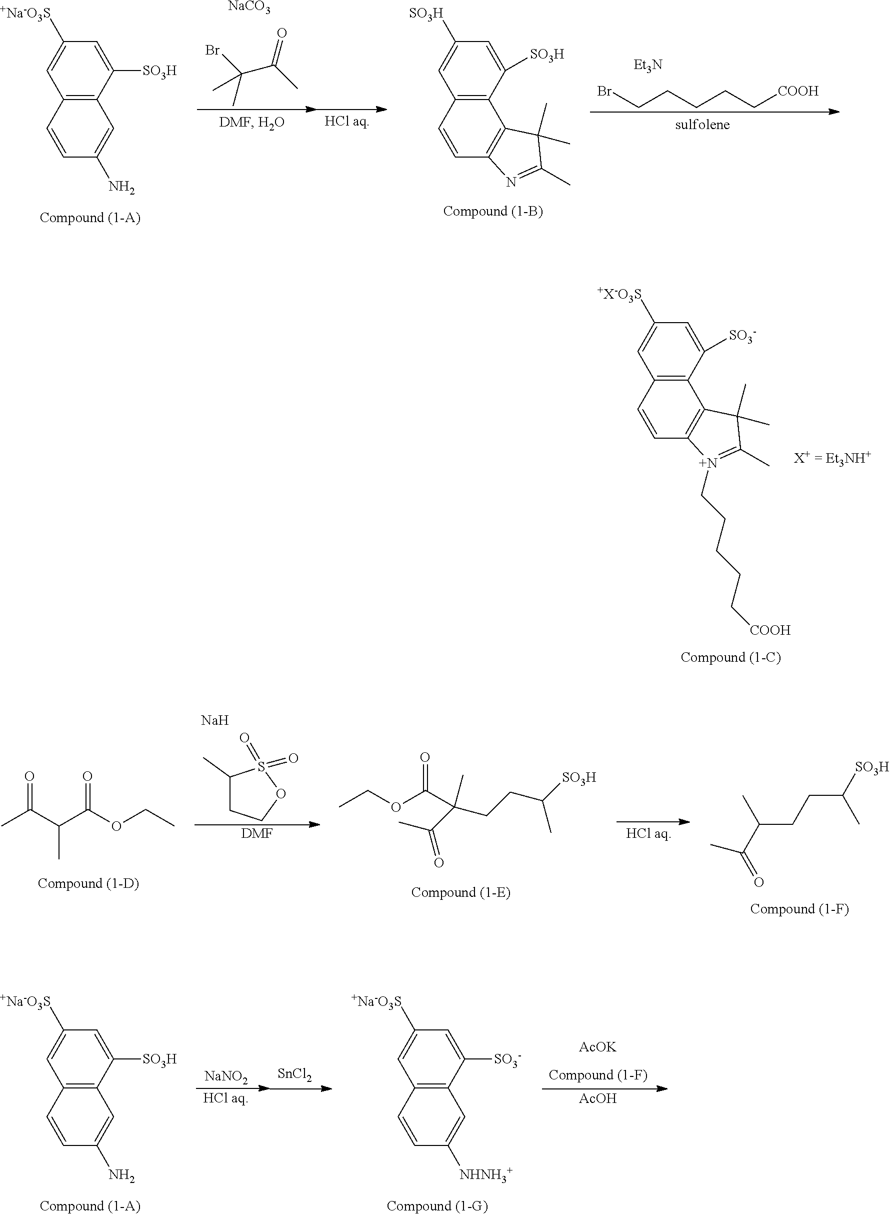

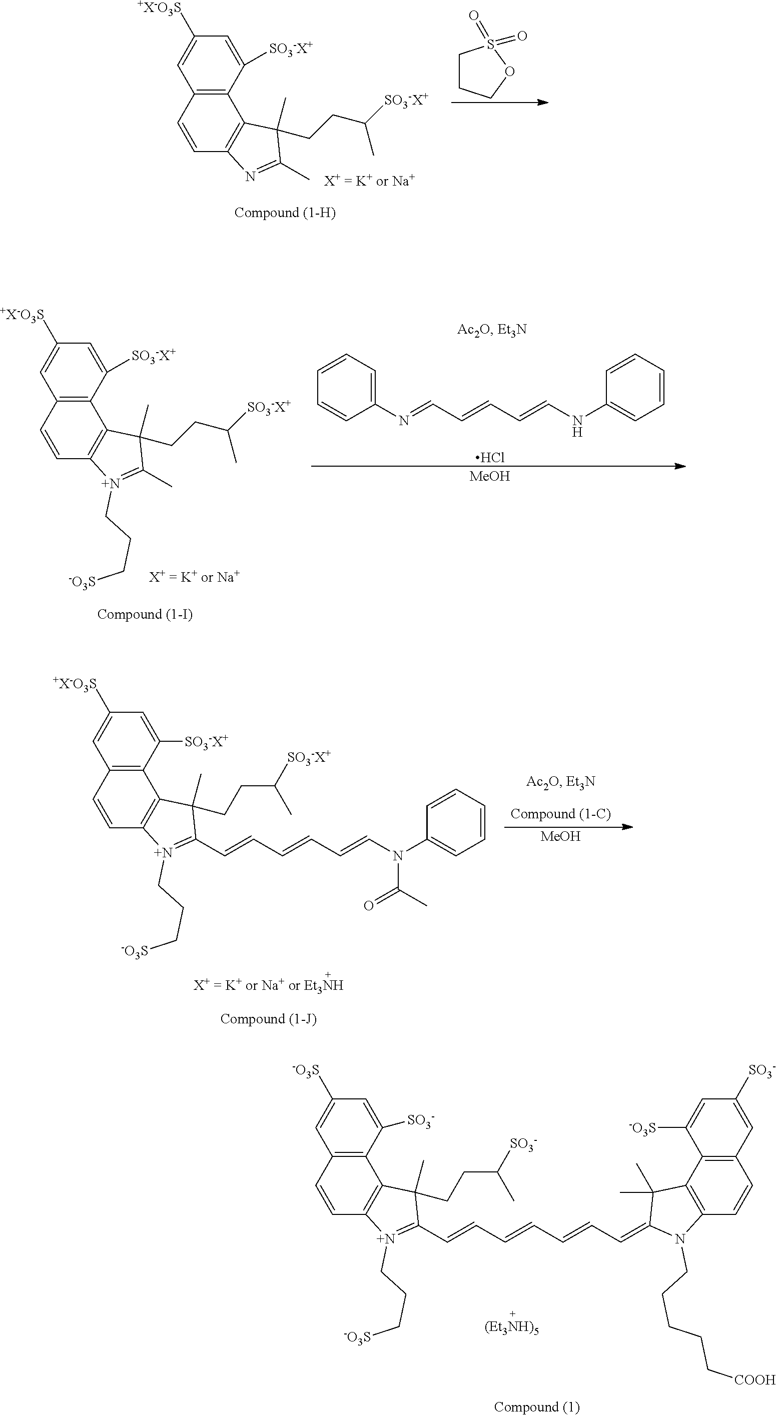

1) Synthesis of compound (1-B)

10 g of a compound (1-A), 30 ml of N,N-dimethylformamide (DMF), 3.3 ml of distilled water, 3.1 g of sodium carbonate, and 7.42 g of 3-bromo-3-methyl-2-butanone were added in a 200 ml three-necked flask, and the resultant mixture was heated and stirred at 90° C. for 12 hours in a nitrogen atmosphere. Then, the solvent was distilled off under reduced pressure, 15 ml of a 10% hydrochloric acid aqueous solution was added thereto, and the resultant mixture was heated and stirred at 90° C. for 12 hours. Then, the solvent was distilled off under reduced pressure, dispersed in methanol, and subjected to filtration. The filtrate was concentrated under reduced pressure, acetone was added to cause precipitation, and the supernatant was removed by decantation. This crude product was purified by reverse phase column chromatography (acetonitrile/water=from 0/100 to 10/90) to obtain 3.8 g of a compound (1-B).

2) Synthesis of compound (1-C)

500 mg of the compound (1-B), 2 ml of sulfolane, 365 mg of 6-bromohexanoic acid, and 0.169 ml of triethylamine (Et₃N) were added in a 50 ml eggplant flask and reacted at 120° C. for 6 hours. Ethyl acetate was added thereto to cause precipitation. The precipitate was purified by reverse phase column chromatography (eluent: acetonitrile/water=from 0/100 to 20/100) to obtain 100 mg of a compound (1-C).

3) Synthesis of compound (1-F)

15 ml of N,N-dimethylformamide and 1.67 g of sodium hydride were added in a nitrogen-substituted 50 ml three-necked flask, and while stirring the resultant mixture, 5.1 ml of a compound (1-D) was added dropwise thereto, and stirring was carried out for a while. Next, 4.1 ml of 2,4-butane sultone was added dropwise thereto, and the resultant mixture was heated and stirred. After stirring at 80° C. for 30 minutes, 5 ml of N,N-dimethylformamide was added thereto, and the resultant mixture was reacted for 4 hours. The solvent was distilled off under reduced pressure, a liquid separation operation was carried out with ethyl acetate and distilled water, and a crude product was extracted with distilled water. 18 ml of a 30% hydrochloric acid aqueous solution was added to the obtained crude product, and the resultant mixture was reacted at 100° C. for 3 hours. Then, the solvent was distilled off under reduced pressure, and purification was carried out by normal phase column chromatography (eluent: ethyl acetate/methanol=from 0/100 to 25/75) to obtain 3 g of a compound (1-F).

4) Synthesis of compound (1-G)

20 g of the compound (1-A) and 120 ml of distilled water were added in a 1 L three-necked flask, and while stirring the resultant mixture, a solution obtained by mixing a 30% hydrochloric acid aqueous solution with 40 ml of distilled water was added dropwise thereto. The mixture was cooled in a salt-ice bath, and a solution obtained by dissolving 4.22 g of sodium nitrite in 80 ml of distilled water was slowly added dropwise while maintaining the temperature at 3° C. or lower, and then the mixture was stirred at 0° C. to 3° C. for 45 minutes. Subsequently, a solution obtained by dissolving 21 g of tin (II) chloride in 60 ml of distilled water and 20 ml of a 30% HCl was slowly added dropwise, and then the mixture was stirred for 40 minutes at 7° C. or lower. Then, isopropanol was added to the reaction solution to generate a precipitate, and then suction filtration was carried out to obtain 15 g of a compound (1-G).

5) Synthesis of compound (1-H)

2.0 g of the compound (1-G), 30 ml of acetic acid (AcOH), 2.1 ml of the compound (1-F), and 1.24 g of potassium acetate (AcOK) were added in a 200 ml eggplant flask and reacted at 140° C. for 1 hour in a nitrogen atmosphere. The temperature was returned to room temperature, 90 ml of ethyl acetate was added, and the generated precipitate was filtered. The filtrate was purified by reverse phase column chromatography (eluent: acetonitrile/water=from 0/100 to 25/75) to obtain 1 g of a compound (1-H).

6) Synthesis of compound (1-I)

200 mg of the compound (1-H), 2 ml of sulfolane, 0.72 ml of 1,3-propane sultone, and 0.142 ml of N-ethyldiisopropylamine were added in a 50 ml eggplant flask and reacted at 120° C. for 1.5 hours. The temperature was returned to room temperature, ethyl acetate was added to generate a precipitate, and the supernatant was removed by decantation. The obtained crude product was purified by reverse phase column chromatography (eluent: acetonitrile/water=0/100) to obtain 152 mg of a compound (1-I).

7) Synthesis of compound (1-J)

5 mg of the compound (1-I), 0.1 ml of dimethyl sulfoxide, and 0.1 ml of MeOH were added in a test tube and subjected to sonication. While stirring, 1.8 mg of glutaconaldehydedianil hydrochloride, 3 μ1 of acetic anhydride (Ac₂O), and 2.4 μl of triethylamine (Et3N) were added thereto, and the resultant mixture was stirred for a while in a nitrogen atmosphere. After the reaction converged, 18 ml of ethyl acetate was added to cause precipitation, and the precipitate was collected by filtration and purified by reverse phase column chromatography (eluent: acetonitrile/water=from 0/100 to 25/75) to obtain 8 mg of a compound (1-J). This reaction and purification were repeated 3 times to obtain 22 mg of the compound (1-J).

8) Synthesis of compound (1)

8 mg of the compound (1-C), 16 mg of the compound (1-J), 0.6 ml of methanol, 8 μl of acetic anhydride, and 6 μl of triethylamine were added to a 50 ml eggplant flask, and the resultant mixture was stirred at room temperature for 16 hours in a nitrogen atmosphere. After adding 2 ml of methanol, 30 ml of ethyl acetate was added to generate a precipitate, and the generated precipitate was filtered and separated. This was purified by preparative HPLC and subjected to lyophilization. After thawing the purified product, methanol and a trace amount of triethylamine were added thereto, the resultant mixture was stirred for 30 minutes, and then the solvent was distilled off by a centrifugal evaporator, followed by drying to obtain 2 mg of a compound (1).

MS (ESI m/z):(M+H⁺)⁺=1,159, (M−H⁺)⁻=1,157

9) Synthesis of labeled antibody (1)

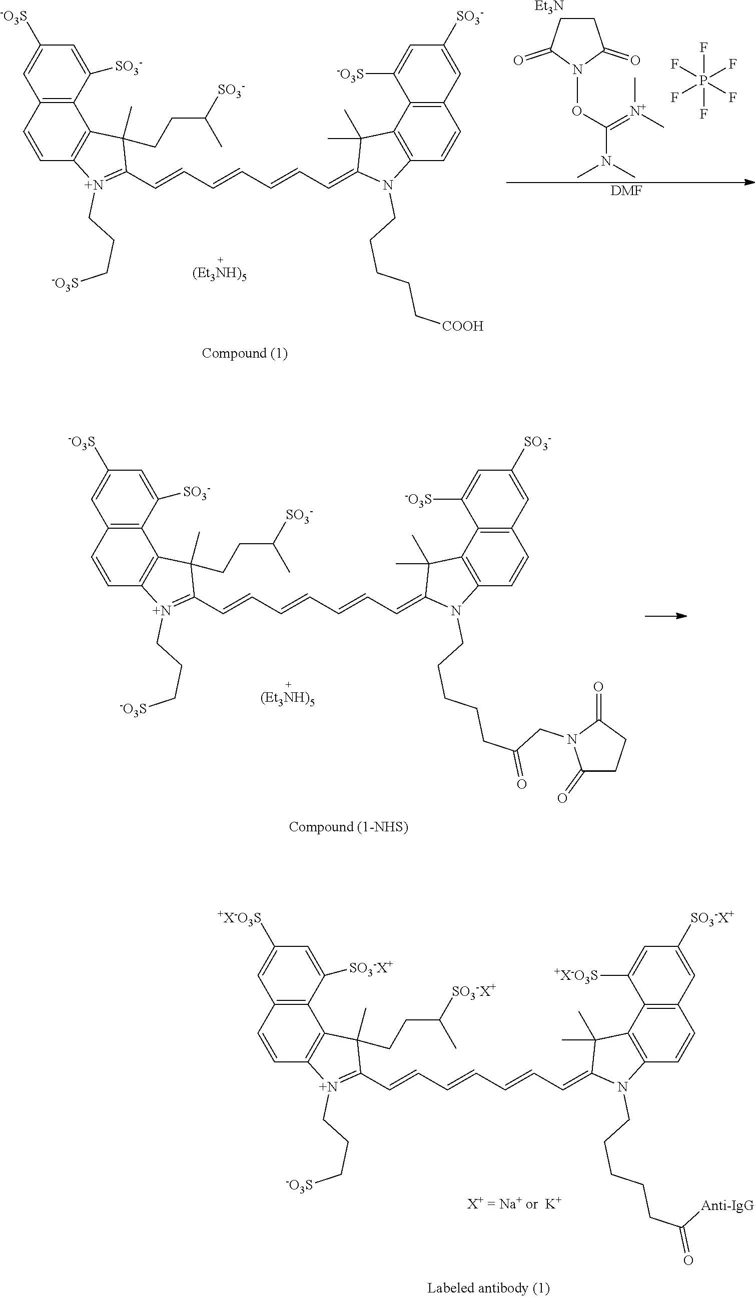

an N,N-dimethylformamide solution obtained by dissolving, 0.28 ml of N,N-dimethylformamide and 0.7 mg of N,N,N′,N′-TETRAMETHYL-O-(N-SUCCINIMIDYL) URONIUM HEXAFLUOROPHOSPHATE, and triethylamine were added to the compound (1) and stirred for 3 hours. Then, ethyl acetate was added, the supernatant was removed, and vacuum drying was carried out to obtain a compound (1-NHS).

217 μl of an anti-rabbit IgG antibody (2.3 mg/ml) and 21.7 μl of a carbonate buffer were added in a microtube, the resultant mixture was shaken and stirred. A dimethyl sulfoxide solution of the compound (1-NHS) was added thereto so that the molar ratio of the compound (1-NHS) to the antibody was 3, and the resultant mixture was shaken and stirred. After being allowed to stand at room temperature for 1 hour, the reaction solution was purified using a gel filtration chromatography column PD10 (manufactured by GE Healthcare Life Sciences) and a PBS solution to obtain a labeled antibody (1).

### Synthesis Example 2

A compound (2) was synthesized based on the following scheme.

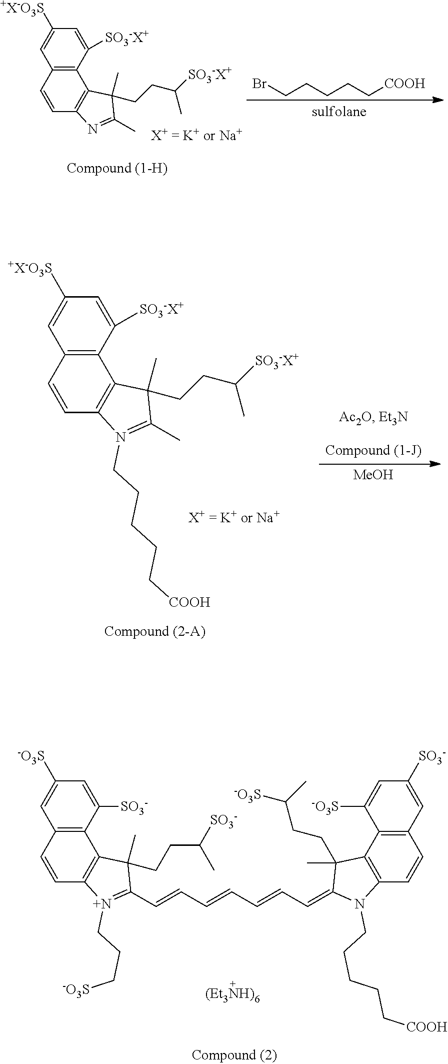

50 mg of the compound (1-H), 341 mg of 6-bromohexanoic acid, 1 ml of sulfolane, and 0.1 ml of distilled water were added in a 50 ml eggplant flask, and the resultant mixture was stirred at 130° C. for 30 minutes. The temperature was returned to room temperature, ethyl acetate was added to cause precipitation, and the supernatant was removed. The precipitate was purified by reverse phase column chromatography (eluent: acetonitrile/water=from 0/100 to 15/85) to obtain 100 mg of a compound (2-A).

A compound (2) was synthesized in the same manner except that the compound (1-C) in the synthesis of the compound (1) was replaced with the compound (2-A).

MS (ESI m/z):(M+H⁺)⁺=1,281, (M−H⁺)⁻=1,279

Synthesis of labeled antibody (2)

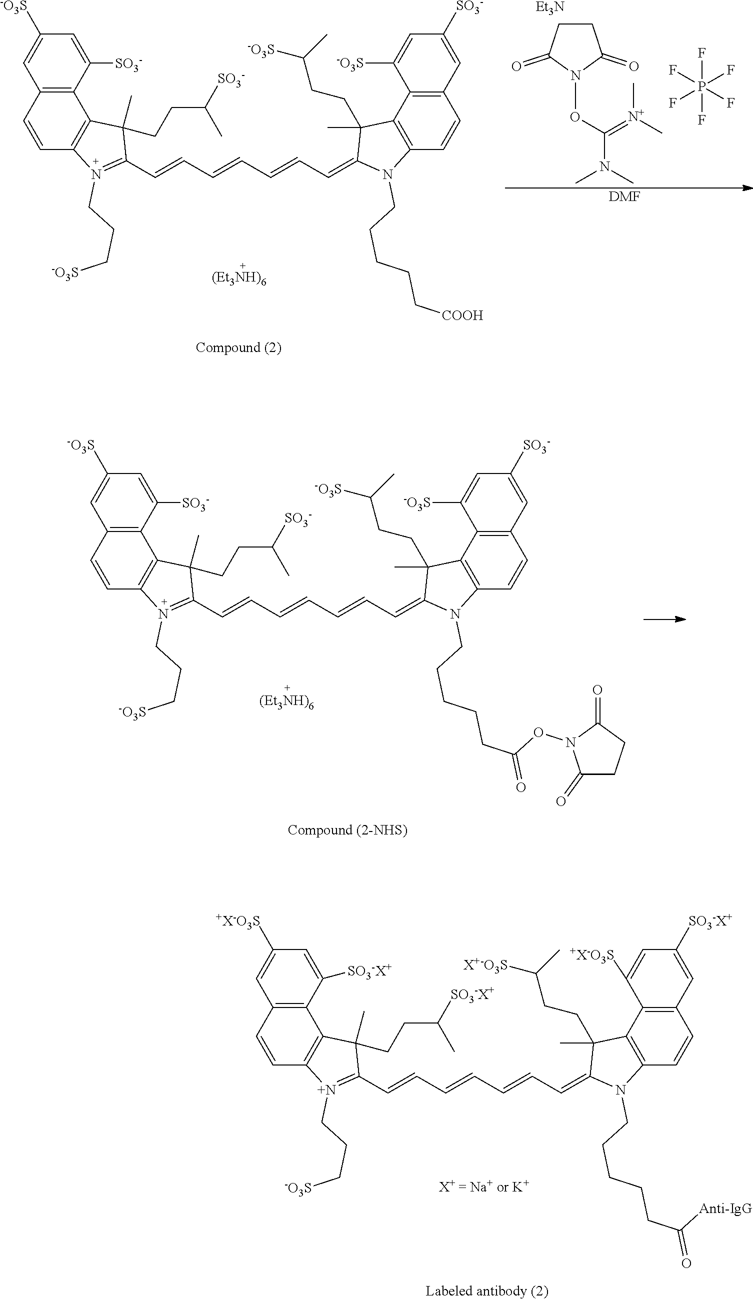

A labeled antibody (2) was synthesized in the same manner except that in the synthesis of the labeled antibody (1), the compound (1) was replaced with the compound (2).

### Synthesis Example 3

A compound (3) was synthesized based on the following scheme.

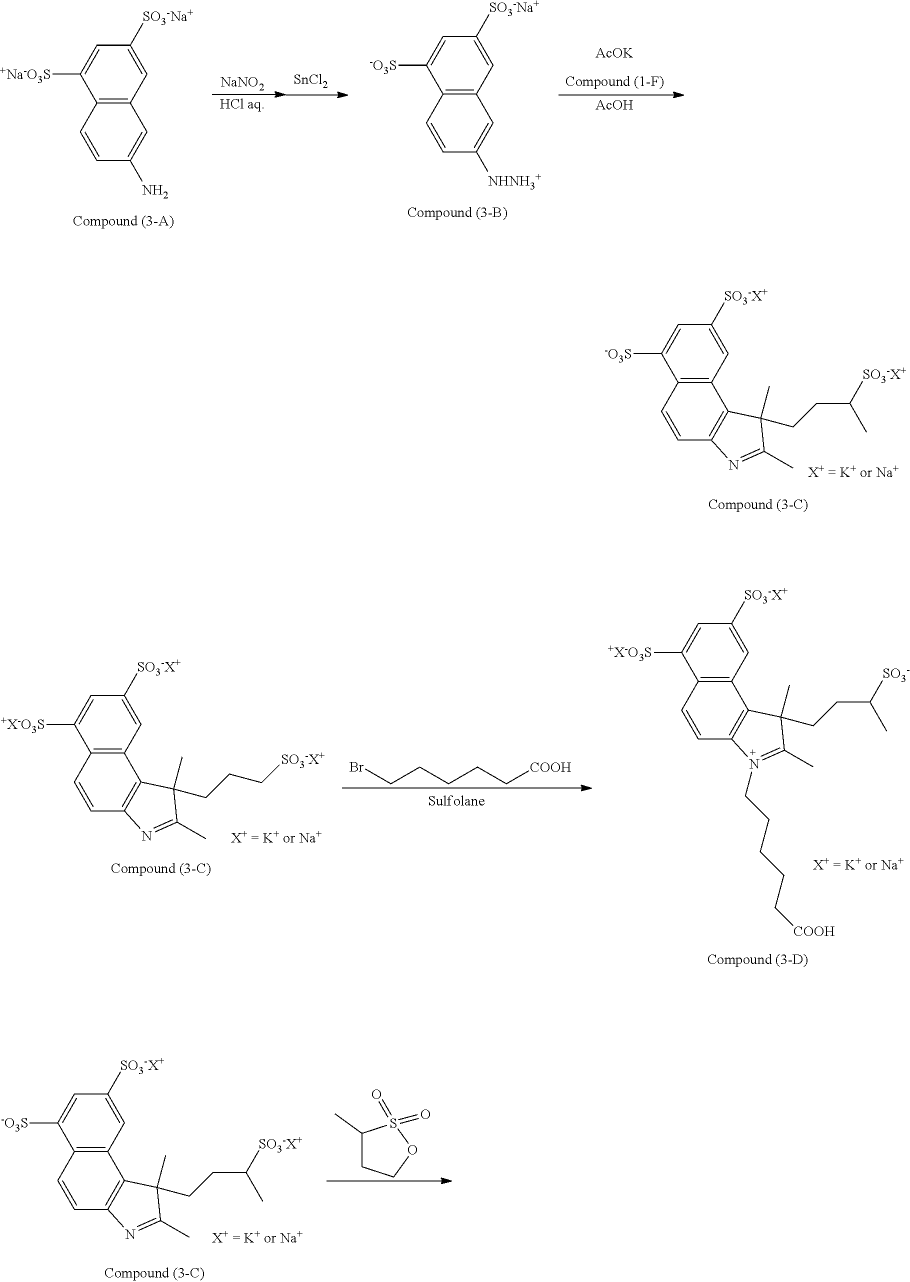

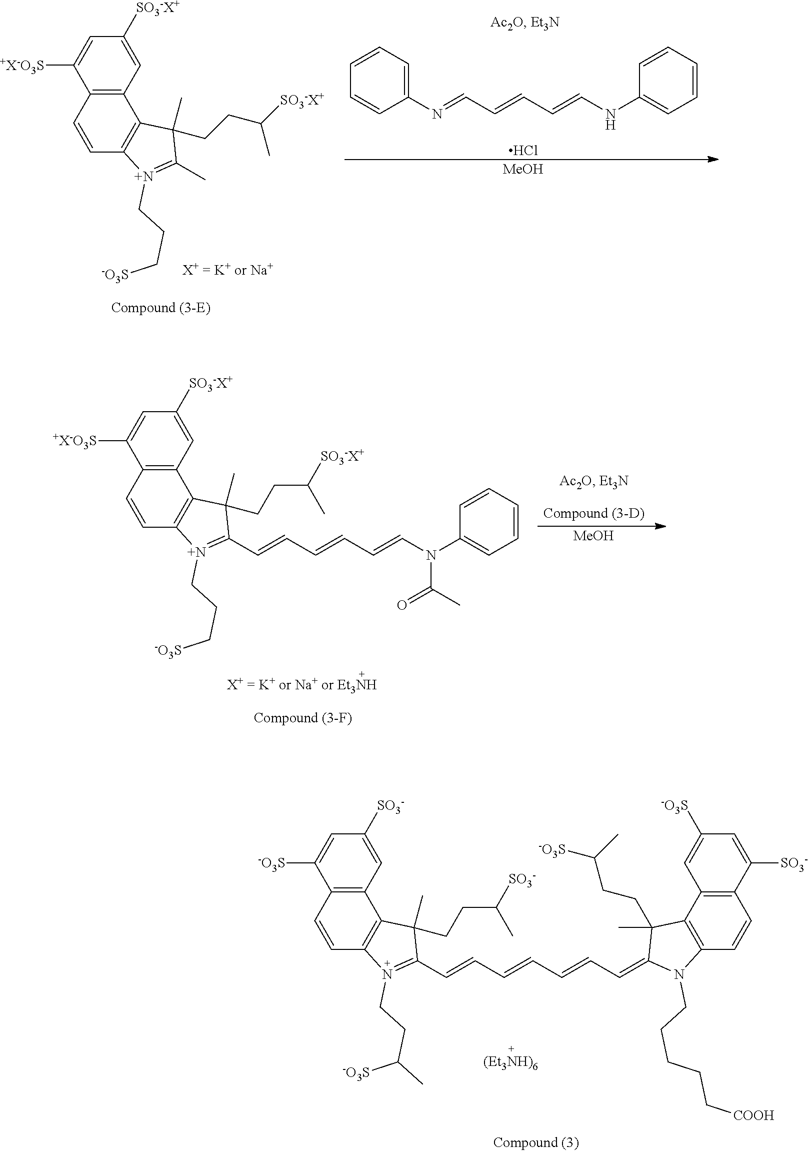

A compound (3-C) was synthesized in the same manner except that in the synthesis of the compound (1-H), the compound (1-A) was replaced with a compound (3-A). In addition, a compound (3-D) was synthesized in the same manner except that in the synthesis of the compound (2-A), the compound (1-H) was replaced with a compound (3-C).

A compound (3) was synthesized in the same manner except that in the synthesis of the compound (1), the compound (1-H) was replaced with the compound (3-C), 1,3-propane sultone was replaced with 2,4-butane sultone, and the compound (1-C) was replaced with the compound (3-D).

MS (ESI m/z):(M+H⁺)⁺=1,295, (M−H⁺)⁻=1,293

Synthesis of labeled antibody (3)

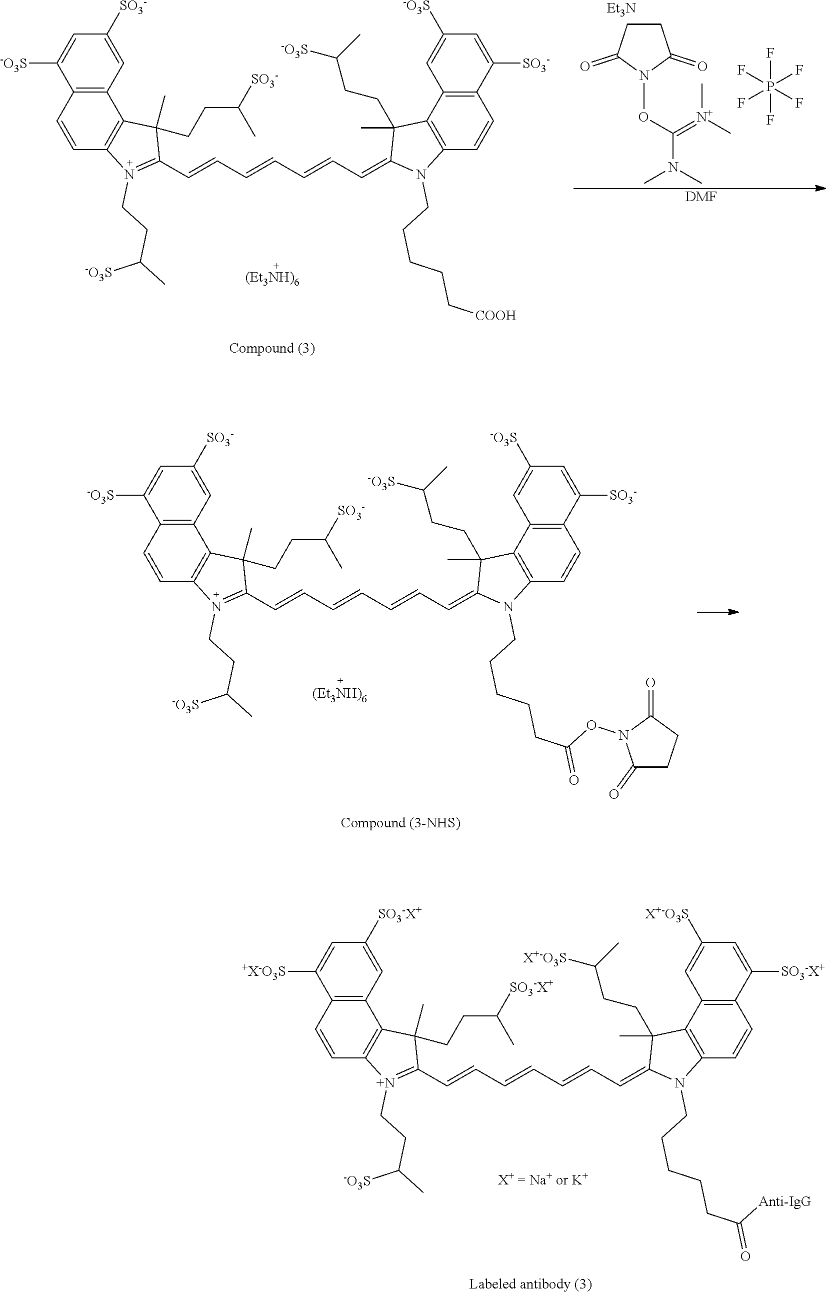

A labeled antibody (3) was synthesized in the same manner except that in the synthesis of the labeled antibody (1), the compound (1) was replaced with the compound (3).

### Synthesis Example 4

A compound (4) was synthesized based on the following scheme.

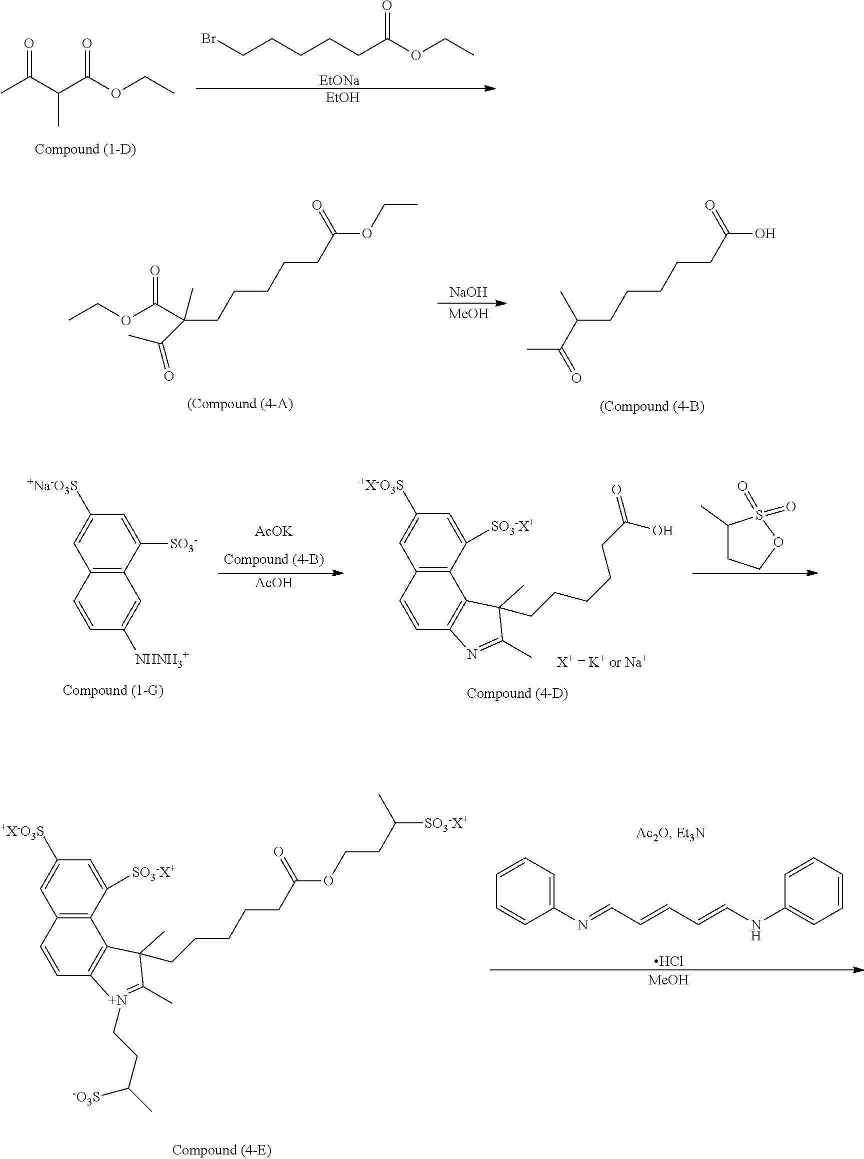

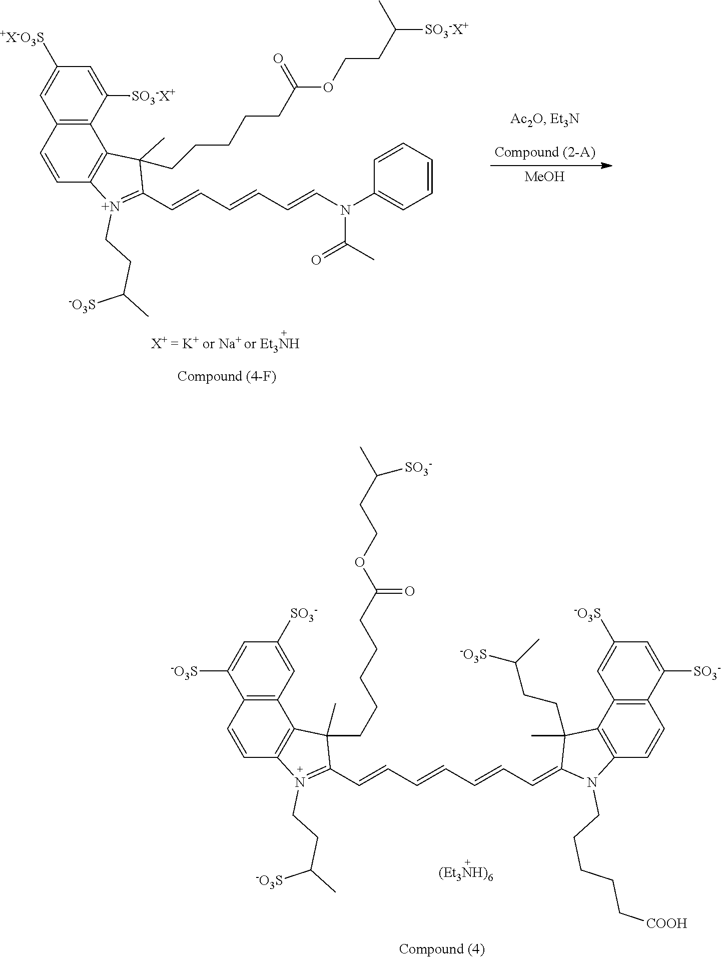

1) Synthesis of compound (4-B)

25 ml of the compound (1-D), 34 ml of ethyl 6-bromohexanoate, 200 ml of ethanol, and 67.4 ml of a 2.68 M sodium ethoxide/ethanol solution were added in a 1 L eggplant flask and reacted at 90° C. for 12 hours. Suction filtration was carried out, the filtrate was concentrated under reduced pressure, 100 ml of a 1 M hydrochloric acid aqueous solution, and 100 ml of chloroform were added thereto, and a liquid separation operation was carried out. An organic layer was removed, and washing was carried out again with 100 ml of chloroform to remove an organic layer. After drying with magnesium sulfate, the supernatant was collected and subjected to concentration under reduced pressure. The obtained crude product was purified by silica gel column chromatography using hexane/ethyl acetate (from 100/0 to 85/15) as an eluent to obtain a compound (4-A).

Next, using the whole amount of the obtained compound (4-A) as it was, a solution obtained by dissolving 300 ml of methanol and 10 g of sodium hydroxide in 100 ml of distilled water was added in a 500 ml three-necked flask, and the resultant mixture was reacted at 90° C. for 12 hours. The reaction mixture was concentrated under reduced pressure, and 25 ml of a 30% hydrochloric acid aqueous solution was added dropwise to bring the pH to 1 or less. After adding 150 ml of ethyl acetate and carrying out a liquid separation operation to remove an organic layer, 100 ml of ethyl acetate was added again to remove an organic layer. After drying with magnesium sulfate, the supernatant was subjected to concentration under reduced pressure to obtain 10 g of a compound (4-B).

2) Synthesis of compound (4-D)

A compound (4-D) was synthesized in the same manner as the method of synthesizing the compound (1-H) except that the compound (1-F) in the synthesis of the compound (1-H) was replaced with the compound (4-B).

3) Synthesis of compound (4-E)

150 mg of the compound (4-D), 10 ml of methanol, and 45 mg of potassium acetate were added in a 50 ml eggplant flask, stirred for 5 minutes, and then subjected to concentration under reduced pressure. Next, 1.5 ml of 2,4-butane sultone and 150 μl of distilled water were added thereto, and the resultant mixture was reacted at 140° C. for 2 hours. The temperature was returned to room temperature, ethyl acetate was added to cause precipitation, and the supernatant was removed. After vacuum drying, purification was carried out by reverse phase column chromatography (eluent: acetonitrile/water=from 0/100 to 25/75) to obtain 40 mg of a compound (4-E).

4) Synthesis of compound (4-F)

A compound (4-F) was synthesized according to the same synthesis method as that of the compound (1-J) except that the compound (1-I) in the synthesis of the compound (1-J) was replaced with the compound (4-E).

5) Synthesis of compound (4)

A compound (4) was synthesized in the same manner as the method of synthesizing the compound (3) except that the compound (3-F) in the synthesis of the compound (3) was replaced with the compound (4-F).

MS (ESI m/z):(M+H⁺)⁺=1,409, (M−H⁺)⁻=1,407

6) Synthesis of labeled antibody (4)

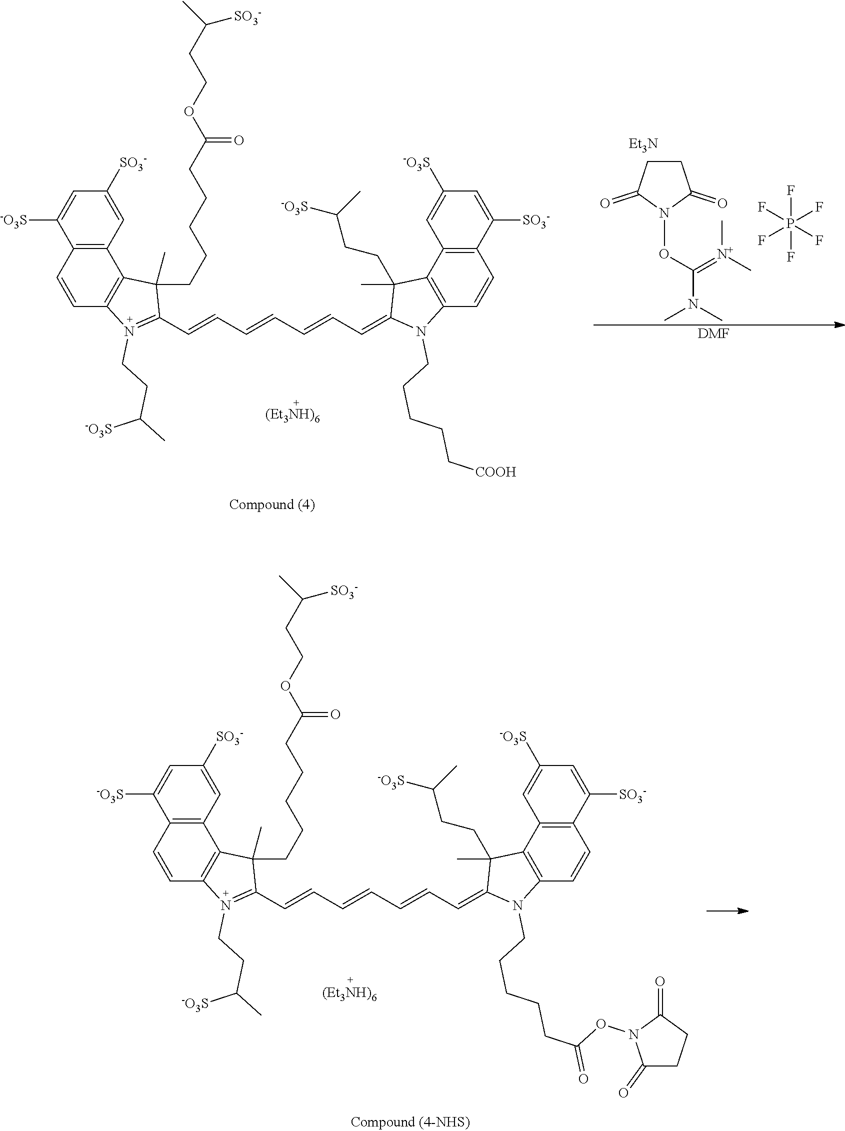

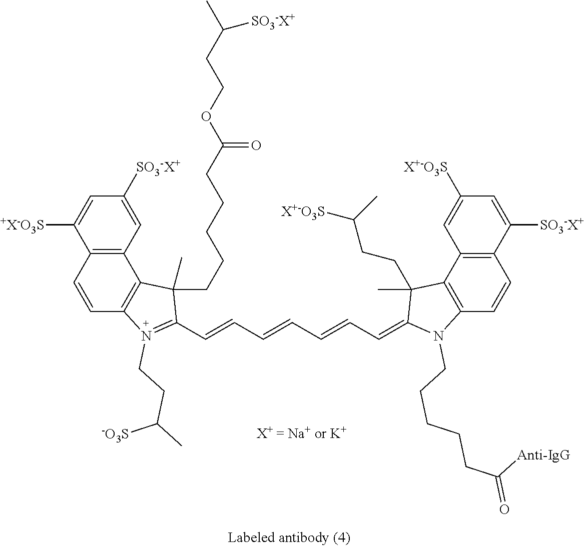

A labeled antibody (4) was synthesized in the same manner except that in the synthesis of the labeled antibody (1), the compound (1) was replaced with the compound (4).

### Synthesis Example 5

A compound (5) was synthesized based on the following scheme.

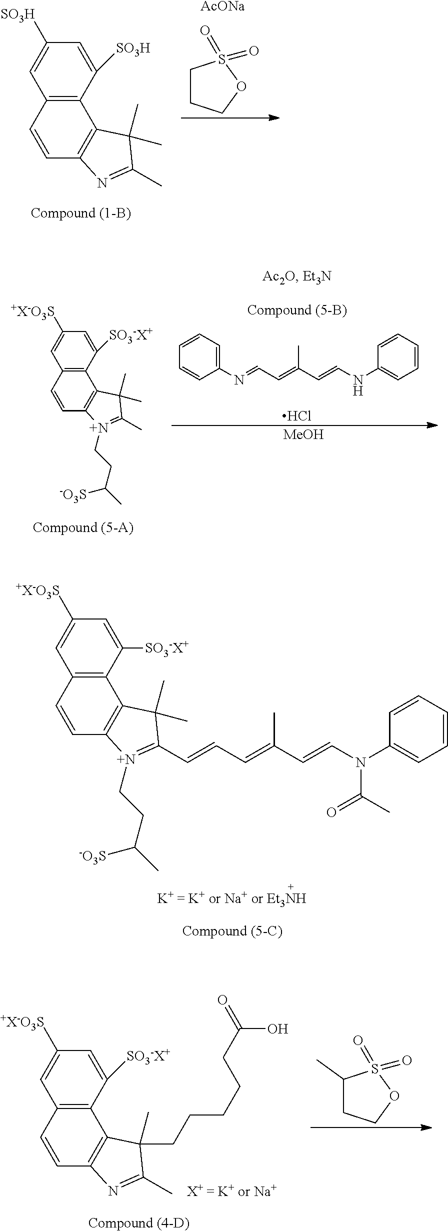

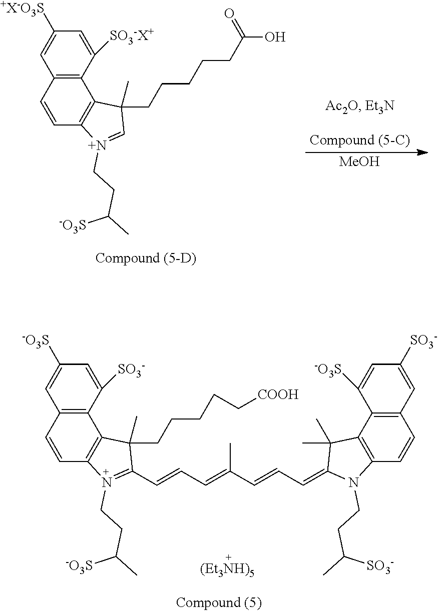

1) Synthesis of compound (5-A)

300 mg of the compound (1-B), 1.51 ml of 2,4-butane sultone, and 0.20 ml of sodium acetate were added in a 10 ml eggplant flask and reacted at 140° C. for 4 hours. The temperature was returned to room temperature, acetone was added to cause precipitation, the precipitate was collected by filtration and then the filtrate was purified by reverse phase column chromatography (eluent: acetonitrile/water=from 0/100 to 5/95) to obtain 100 mg of a compound (5-A).

2) Synthesis of compound (5-C)

A compound (5-C) was synthesized in the same manner as the method of synthesizing the compound (1-J) except that in the synthesis of compound (1-J), the compound (1-I) was replaced with a compound (5-A), glutaconaldehydedianil hydrochloride was replaced with the compound (5-B).

3) Synthesis of compound (5-D)

150 mg of the compound (4-D), 10 ml of methanol, and 45 mg of potassium acetate were added in a 50 ml eggplant flask, stirred for 5 minutes, and then subjected to concentration under reduced pressure. Next, 1.35 ml of 2,4-butane sultone and 150 μl of distilled water were added, and the mixture was reacted at 110° C. for 1 hour. The temperature was returned to room temperature, ethyl acetate was added to cause precipitation, and the supernatant was removed. After vacuum drying, purification was carried out by reverse phase column chromatography (eluent: acetonitrile/water=from 0/100 to 15/85) to obtain 55 mg of a compound (5-D).

4) Synthesis of compound (5)

A compound (5) was synthesized in the same manner as the method of synthesizing the compound (2) except that the compound (1-J) in the synthesis of the compound (2) was replaced with the compound (5-C) and the compound (2-A) was replaced with the compound (5-D).

MS (ESI m/z):(M+H⁺)⁺=1,187, (M−H⁺)⁻=1,185

5) Synthesis of labeled antibody (5)

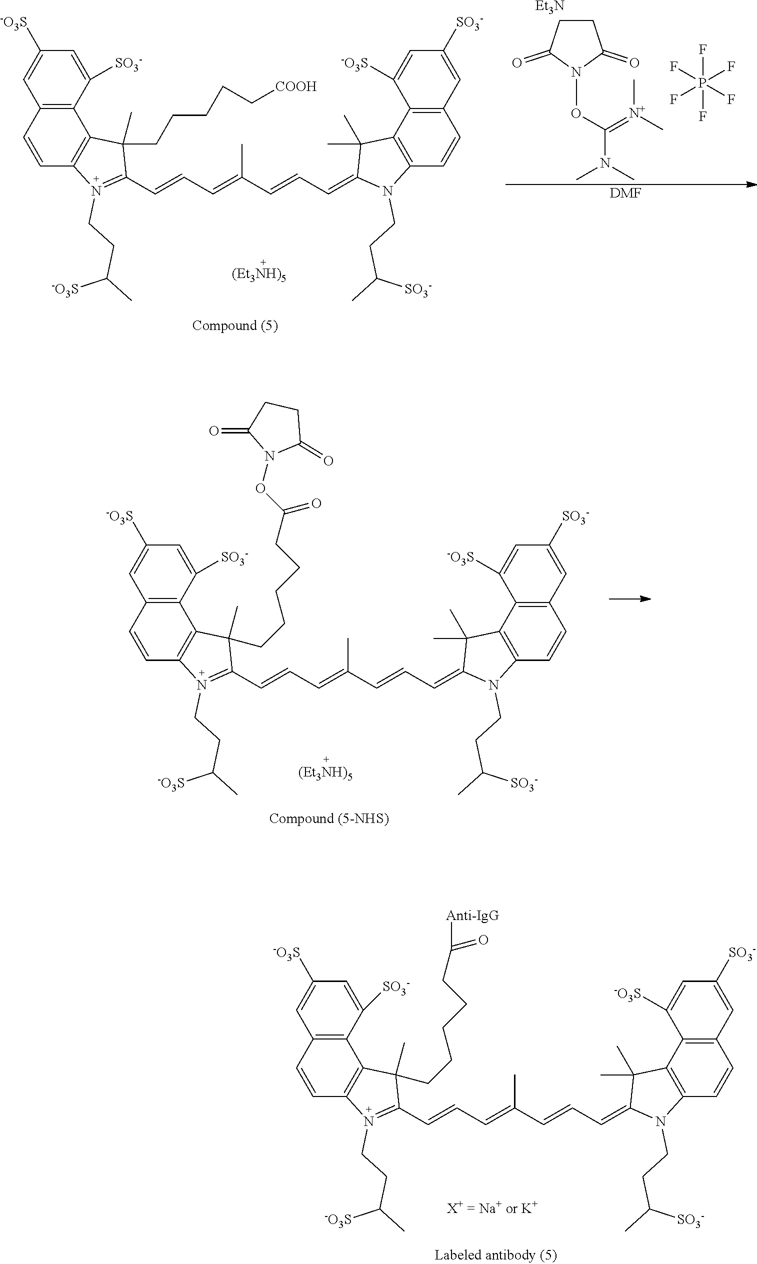

A labeled antibody (5) was synthesized in the same manner except that in the synthesis of the labeled antibody (1), the compound (1) was replaced with the compound (5).

### Synthesis Example 6

A compound (6) was synthesized based on the following scheme.

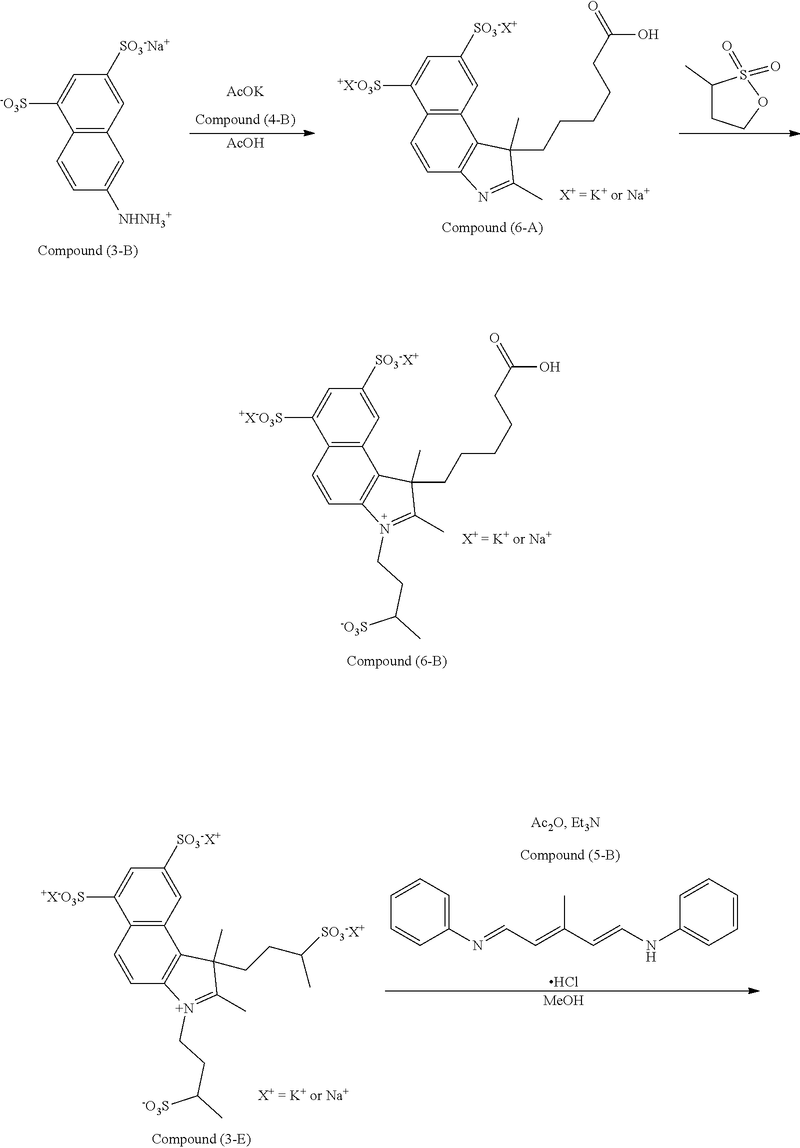

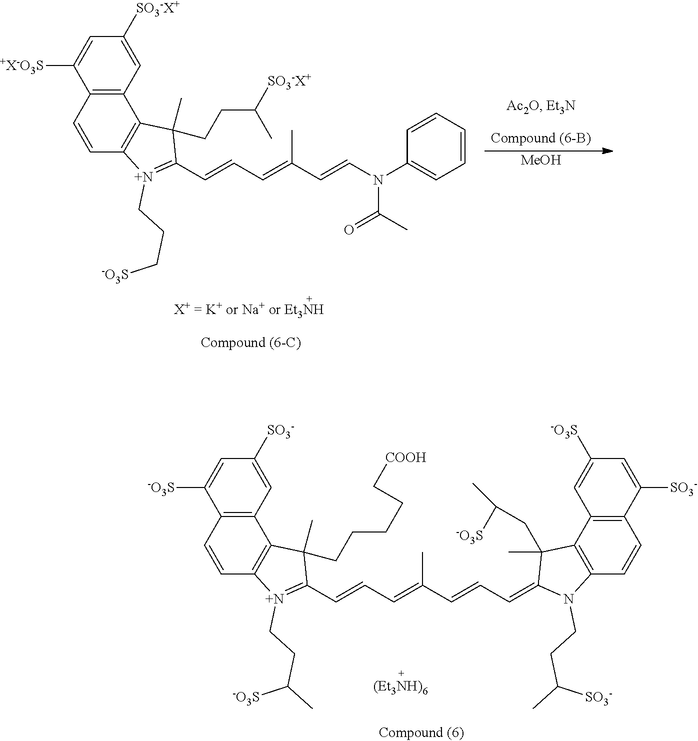

1) Synthesis of compound (6-A)

A compound (6-A) was synthesized in the same manner as the method of synthesizing the compound (4-D) except that the compound (1-G) in the synthesis of the compound (4-D) was replaced with a compound (3-B).

2) Synthesis of compound (6-B)

A compound (6-B) was synthesized in the same manner as the method of synthesizing the compound (3-E) except that the compound (3-C) in the synthesis of the compound (3-C) was replaced with the compound (6-A).

3) Synthesis of compound (6-C)

A compound (6-C) was synthesized in the same manner as the method of synthesizing the compound (5-C) except that the compound (5-A) in the synthesis of the compound (5-C) was replaced with the compound (3-E).

4) Synthesis of compound (6)

A compound (6) was synthesized in the same manner as the method of synthesizing the compound (5) except that in the synthesis of the compound (5), the compound (5-C) was replaced with the compound (6-C) and the compound (5-D) was replaced with the compound (6-B).

MS (ESI m/z):(M+H⁺)⁺=1,309, (M−H⁺)⁻=1,307

5) Synthesis of labeled antibody (6)

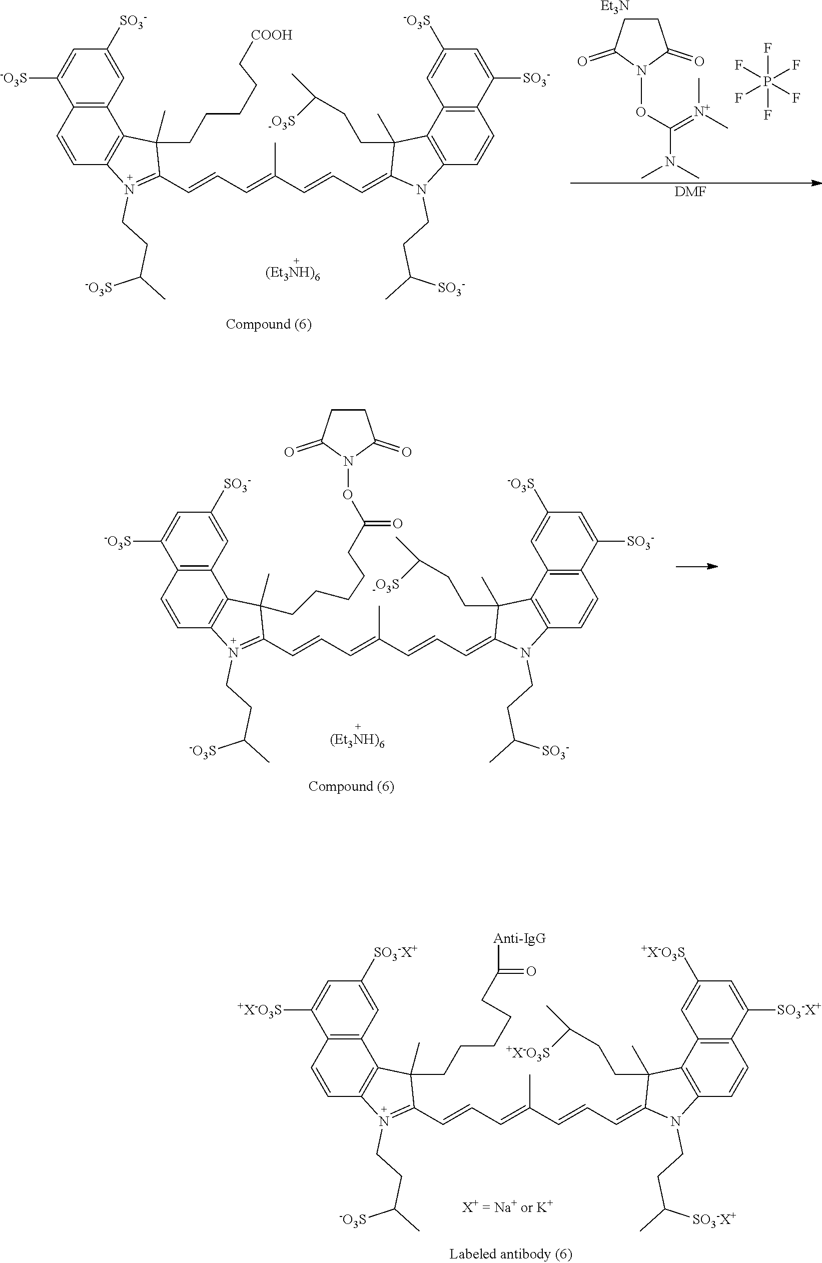

A labeled antibody (6) was synthesized in the same manner except that in the synthesis of the labeled antibody (1), the compound (1) was replaced with the compound (6).

### Example 1

The following characteristics were evaluated for each of the labeled antibodies synthesized as described above, and the obtained results are shown in Table 1.

[Evaluation of Fluorescence Intensity]

For the solution of each of the labeled antibodies prepared as described above, the integrated value of the fluorescence intensity in the fluorescence wavelength range of 810 nm to 840 nm was calculated by using a spectroscopic fluorescence intensity meter (product name: RF-5300, manufactured by Shimadzu Corporation) with excitation light of 785 nm and unified the exposure conditions. Using the integrated value of the fluorescence intensity of the comparative labeled antibody (1) in the fluorescence wavelength range of 810 nm to 840 nm as the reference value, the ratio to this reference value (the integrated value of the fluorescence intensity in the fluorescence wavelength range of 810 nm to 840 nm/the reference value) was calculated, and then, the evaluation was made based on the following evaluation standards.

In the present test, it is determined that a compound has passed the evaluation of the fluorescence intensity in a case where the compound has a rank “B” or higher.

Evaluation Standards for Fluorescence Intensity

A: The ratio of fluorescence intensity to the reference value is more than 2 times.

B: The ratio of fluorescence intensity to the reference value is 1.2 times or more and less than 2 times.

C: The ratio of fluorescence intensity to the reference value is 0.9 times or more and less than 1.2 times.

D: The ratio of fluorescence intensity to the reference value is less than 0.9 times.

TABLE 1

| No. | Labeled antibody | Fluorescence intensity |
|---|---|---|
| 101 | Compound (1) - IgG | B |
| 102 | Compound (2) - IgG | A |
| 103 | Compound (3) - IgG | A |
| 104 | Compound (4) - IgG | A |
| 105 | Compound (5) - IgG | B |
| 106 | Compound (6) - IgG | A |
| c01 | Comparative compound (1) - IgG | — (Reference) |
| c02 | Comparative compound (2) - IgG | D |
| c03 | Comparative compound (3) - IgG | D |

(Note in table)

In the column of Labeled antibody, each labeled antibody (Z) is denoted as Compound (Z)-IgG. Further, each comparative labeled antibody (Z) is denoted as Comparative compound (Z)-IgG. Here, Z means the number of each compound.

From the results in Table 1 above, the following points can be seen.

In the comparative compound (1), R¹ to R⁴ are all methyl groups, and the structure is not a structure regulated by the present invention. The fluorescence intensity of the comparative labeled antibody (1) using this comparative compound (1) is low (No. c01).

The comparative compound (2) is not a compound regulated by the present invention in that the total of n1 to n4 in the compound represented by Formula (2) is 2, the number of SO₃⁻X⁺ groups is small, and the naphthalene ring is contained. The fluorescence intensity of the labeled antibody using this comparative compound (2) is low as compared with the comparative labeled antibody (1) (No. c02).

Further, the comparative compound (3) is not a compound defined by the present invention in that the total of n1 to n4 in the compound represented by Formula (2) is 2, the number of SO₃⁻X⁺ groups is small, the naphthalene ring is contained, and further, any one of the provisos of the conditions (I) and (II) regulated by the present invention is not satisfied although it has a structure in which at least one of R¹ to R⁴ is a carboxyalkyl group. The fluorescence intensity of the labeled antibody using this comparative compound (3) is also low as compared with the comparative labeled antibody (1) (No. c03).

On the other hand, all the labeled antibodies of the compounds (1) to (6), regulated by the present invention have a fluorescence intensity of 1.2 times or more with respect to the fluorescence intensity of the comparative labeled antibody (1) and exhibit an excellent fluorescence intensity (No. 101 to 106 with respect to No. c01).

As described above, since the fluorescently labeled biological substance using the compound according to the embodiment of the present invention represented by Formula (2) has an excellent fluorescence intensity with respect to the excitation light source of 785 nm, it can be suitably used in fluorescence labelling such as multicolor WB, the general-purpose properties or convenience of which can be greatly improved.

Further, the fluorescently labeled biological substance, which uses the compound according to the embodiment of the present invention represented by Formula (1), has an excellent fluorescence intensity with respect to the excitation light source of 685 nm, similarly to the fluorescently labeled biological substance, which uses the compound according to the embodiment of the present invention represented by Formula (2). It can be suitably used in fluorescence labelling such as multicolor WB, the general-purpose properties or convenience of which can be greatly improved.

The present invention has been described together with the embodiments of the present invention. However, the inventors of the present invention do not intend to limit the present invention in any part of the details of the description unless otherwise specified, and it is conceived that the present invention should be broadly construed without departing from the spirit and scope of the invention shown in the attached “WHAT IS CLAIMED IS”.

What is claimed is:

1. A compound represented by Formula (1) or Formula (2), 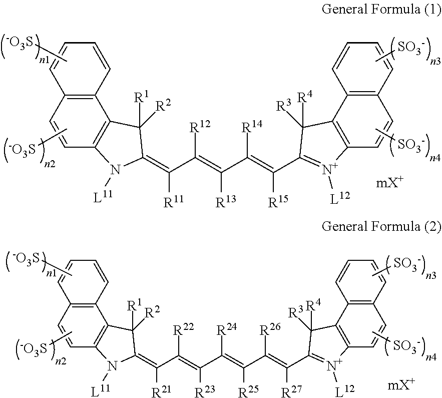
in the formulae, R¹ to R⁴ represent an alkyl group which may have a substituent, provided that at least one of R¹ to R⁴ is a sulfoalkyl group and in a case where the sulfoalkyl group has no substituent other than the sulfo group, at least one of the alkyl groups having only the sulfo group is a branched sulfoalkyl group, R¹ and R² may be linked to each other to form a ring, and R³ and R⁴ may be linked to each other to form a ring,
R¹¹ to R¹⁵ and R²¹ to R²⁷ represent a hydrogen atom, an alkyl group, or an aryl group,
provided that in a case where at least one of R¹ to R⁴ is a carboxyalkyl group or an alkyl group having a substituent capable of being bonded to a biological substance, the following (I) or (II) is satisfied,
(I) in Formula (1), at least one of R¹¹ to R¹⁵ is an alkyl group or an aryl group and in Formula (2), at least one of R²¹ to R²⁷ is an alkyl group or an aryl group or
(II) at least one of R¹ to R⁴, which does not correspond to the carboxyalkyl group or the alkyl group having a substituent capable of being bonded to a biological substance, is an alkyl group having a sulfoalkyl group through a single bond or a linking group,
L¹¹ and L¹² represent an alkyl group which may have a substituent,
n1 to n4 are an integer of 0 to 2, where n1+n2≥1, n3+n4≥1, and n1+n2+n3+n4≥3 are satisfied,
m=n1+n2+n3+n4−1 is satisfied,
X⁺ represents a monovalent cation, and
at least one of R¹ to R⁴, R¹³, L¹¹, or L¹² in Formula (1) and at least one of R¹ to R⁴, R²⁴, L¹¹, or L¹² in Formula (2) have a carboxy group or a substituent capable of being bonded to a biological substance.

2. The compound according to claim 1,
wherein the compound is represented by any one of Formulae (1-1) to (1-6) or Formulae (2-1) to (2-6), 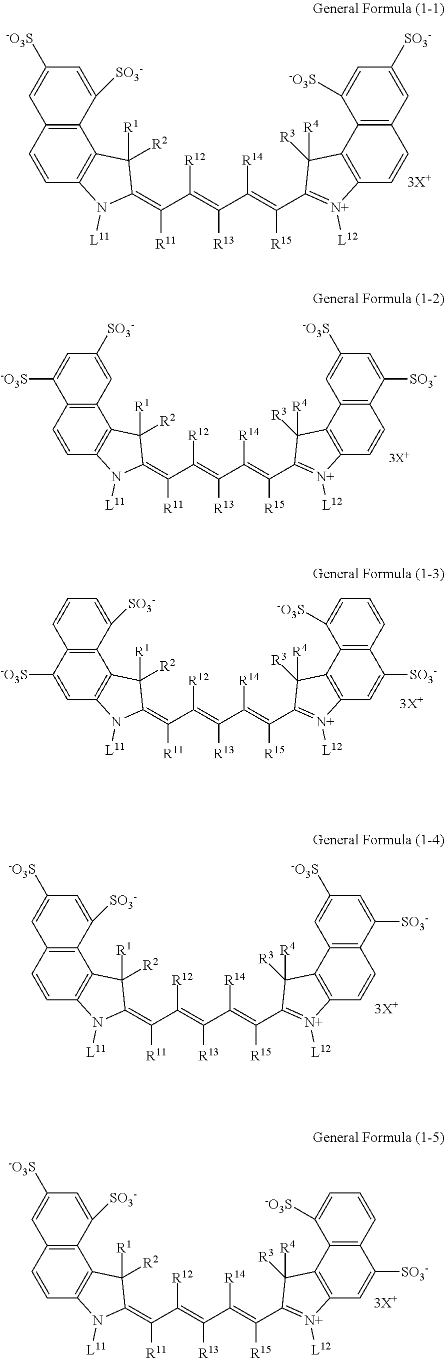 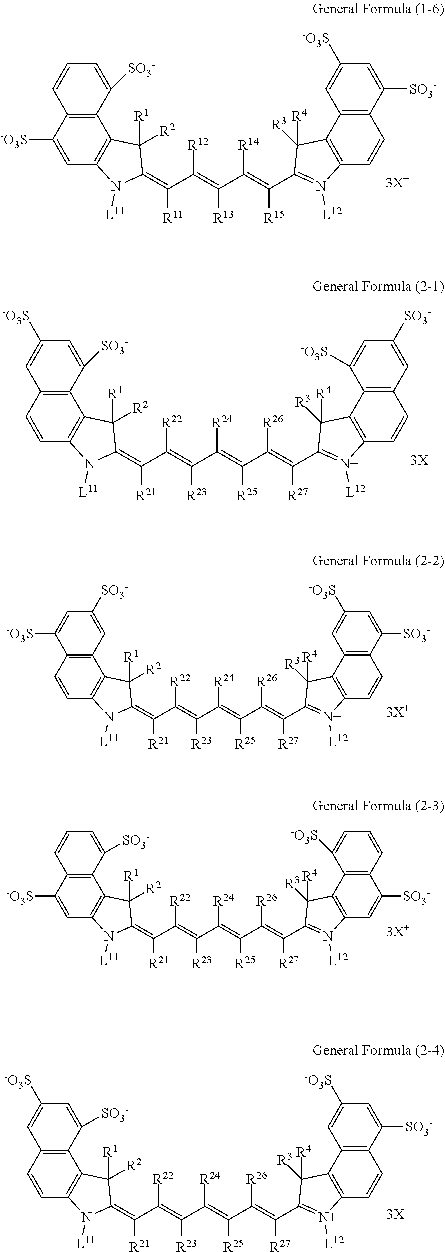 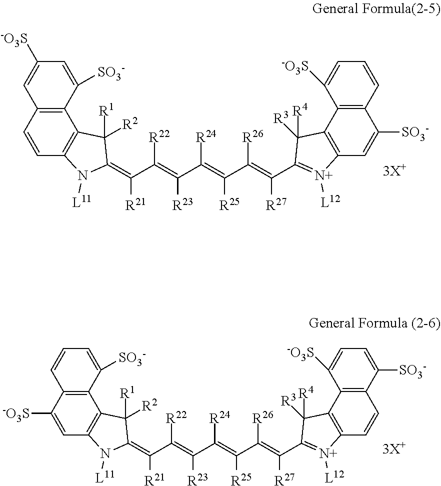
in the formulae, R¹ to R⁴, R¹¹ to R¹⁵, R²¹ to R²⁷, L¹¹, L¹², and X⁺ are respectively synonymous with R¹ to R⁴, R¹¹ to R¹⁵, R²¹ to R²⁷, L¹¹, L¹², and X⁺ in Formula (1) and Formula (2).

3. The compound according to claim 2,
wherein the compound is represented by any one of Formula (1-1), Formula (1-2), Formula (2-1), or Formula (2-2).

4. The compound according to claim 1,
wherein R¹ to R⁴ are an alkyl group which may have, as a substituent, a group selected from an alkoxy group, a carboxy group, an alkoxycarbonyl group, an acyloxy group, an aminocarbonyl group, an acylamino group, a sulfo group, and a phosphono group, and
L¹¹ and L¹² are an alkyl group having, as a substituent, a group selected from an alkoxy group, a carboxy group, an alkoxycarbonyl group, an acyloxy group, an aminocarbonyl group, an acylamino group, a sulfo group, and a phosphono group,
provided that at least one of R¹ to R⁴ is a sulfoalkyl group and in a case where the sulfoalkyl group has no substituent other than the sulfo group, at least one of the alkyl groups having only the sulfo group is a branched sulfoalkyl group.

5. The invention according to claim 1,
wherein R¹¹, R¹², R¹⁴, R¹⁵, R²¹ to R²³, and R²⁵ to R²⁷ are a hydrogen atom, and
R¹³ and R²⁴ are a hydrogen atom or an alkyl group.

6. The invention according to claim 1,
wherein at least one of R¹ or R² is an alkyl group having a substituent, and
at least one of R³ or R⁴ is an alkyl group having a substituent,
provided that at least one of R¹ to R⁴ is a sulfoalkyl group and in a case where the sulfoalkyl group has no substituent other than the sulfo group, at least one of the alkyl groups having only the sulfo group is a branched sulfoalkyl group.

7. The compound according to claim 1,
wherein L¹¹ and L¹² are alkyl groups having at least one of a carboxy group and a sulfo group as a substituent.

8. The compound according to claim 1,
wherein the at least one of R¹ to R⁴, R¹³, L¹¹, or L¹² in Formula (1) and the at least one of R¹ to R^{4,} _{R}²⁴, L¹¹, or L¹² in Formula (2) have a substituent capable of binding to an antibody.

9. A fluorescently labeled biological substance that is obtained by bonding between the compound according to claim 1 and a biological substance.

10. The fluorescently labeled biological substance according to claim 9,
wherein the biological substance is any one of a protein, an amino acid, a nucleic acid, a sugar chain, a lipid, or a phospholipid.
